(19) United States
(12) Patent Application Publication
(10) Pub. No.: US 2022/0170908 A1
(43) Pub. Date: Jun. 2, 2022

(54) COMPOSITIONS AND METHODS FOR CHARACTERIZING AND TREATING ALZHEIMERS DISEASE

(71) Applicants: Anton WYSS-CORAY, Stanford, CA (US); David GATE, Stanford, CA (US); Mark DAVID, Stanford, CA (US); Naresha SALIGRAMA, Stanford, CA (US); David A. CASIMIR, Stanford, CA (US); The Board of Trustees of the Leland Standford Junior Inivesity, Stanford, CA (US); U.S Government as represented by The Department of Veterans Affair, Washibton, DC (US)

(72) Inventors: Anton Wyss-Coray, Stanford, CA (US); David Gate, Stanford, CA (US); Mark Davis, Stanford, CA (US); Naresha Saligrama, Stanford, CA (US)

(21) Appl. No.: 17/442,387

(22) PCT Filed: Mar. 25, 2020
(86) PCT No.: PCT/US20/24603

§ 371 Date: Sep. 23, 2021

Related U.S. Application Data
(60) Provisional application No. 62/823,980, filed on Mar. 26, 2019.

(51) Int. Cl. G01N 33/50; G01N 33/68; A61K 35/17; C07K 14/725

(52) U.S. Cl. CPC G01N 33/505; G01N 33/6896; G01N 2800/2821; C07K 14/7051; G01N 2333/522; A61K 35/17

(57) ABSTRACT

Provided herein am compositions and methods for characterizing and treating neurodegenerative disease. In particular, provided herein are compositions and methods for measuring T cell markers associated with Alzheimer's disease.

This application claims priority to U.S. provisional patent application Ser. No. 62/823,980, filed Mar. 26, 2019, which is incorporated herein by reference in its entirety.

### STATEMENT REGARDING FEDERALLY SPONSORED RESEARCH OR DEVELOPMENT

This invention was made with Government support under contract AI057229 and contract AG045034 awarded by the National Institutes of Health. The Government has certain rights in the invention.

### FIELD OF THE DISCLOSURE

Provided herein are compositions and methods for characterizing and treating neurodegenerative disease. In particular, provided herein are compositions and methods for measuring T cell markers associated with Alzheimer's disease.

### BACKGROUND OF THE DISCLOSURE

Alzheimer's disease (AD) is a chronic neurodegenerative disease that usually starts slowly and worsens over time. It is the cause of 60-70% of cases of dementia. The most common early symptom is difficulty in remembering recent events (short-term memory loss). As the disease advances, symptoms can include problems with language, disorientation (including easily getting lost), mood swings, loss of motivation, not managing self-care, and behavioral issues. As a person's condition declines, they often withdraw from family and society. Gradually, bodily functions are lost, ultimately leading to death. Although the speed of progression can vary, the typical life expectancy following diagnosis is three to nine years.

The cause of Alzheimer's disease is poorly understood. About 70% of the risk is believed to be genetic with many genes usually involved. Other risk factors include a history of head injuries, depression, or hypertension. The disease process is associated with plaques and tangles in the brain. A probable diagnosis is based on the history of the illness and cognitive testing with medical imaging and blood tests to rule out other possible causes. Initial symptoms are often mistaken for normal ageing. Examination of brain tissue is needed for a definite diagnosis.

No treatments stop or reverse its progression, though some may temporarily improve symptoms. Affected people increasingly rely on others for assistance, often placing a burden on the caregiver; the pressures can include social, psychological, physical, and economic elements. Exercise programs may be beneficial with respect to activities of daily living and can potentially improve outcomes. Behavioral problems or psychosis due to dementia are often treated with antipsychotics, but this is not usually recommended, as there is little benefit with an increased risk of early death.

In 2015, there were approximately 29.8 million people worldwide with AD. It most often begins in people over 65 years of age, although 4% to 5% of cases are early-onset Alzheimer's which begin before this. It affects about 6% of people 65 years and older. In 2015, dementia resulted in about 1.9 million deaths. In developed countries, AD is one of the most financially costly diseases.

Additional methods for diagnosing and treating AD are needed.

### SUMMARY OF THE DISCLOSURE

Provided herein are compositions and methods for characterizing and treating neurodegenerative disease. In particular, provided herein are compositions and methods for measuring T cell markers associated with Alzheimer's disease.

Alzheimer's disease (AD) is an incurable neurodegenerative disorder in which neuroinflammation is increasingly recognized to play a critical function. While innate inflammation has been implicated in AD, little is known about the contribution of the adaptive immune response. Described herein are experiments testing peripheral blood (e.g., mononuclear cells) combined with unbiased discovery and machine learning techniques that identified an immunologic signature of AD characterized, for example, by increased numbers of CD8⁺ T effector memory CD45RA⁺ (T_{EMRA}) cells. Levels of the brain homing chemokine C-X-C motif ligand 9 (CXCL9) were significantly higher in AD patient plasma and peripheral CD8⁺ T cell numbers were strongly associated with cognition. It was further determined that CD8⁺ T_{EMRA} cells were also present in patient cerebrospinal fluid (CSF) and T cell receptor (TCR) sequencing indicated their clonal expansion, supporting antigen specificity of these adaptive immune cells. These results reveal a blood-CSF adaptive immune response in AD and demonstrate clonal, antigen-experienced T cells patrolling the intrathecal space of brains affected by age-related neurodegeneration. These results provide assays for characterizing subject with symptoms of neurodegenerative disease to distinguish between AD, other neurodegenerative diseases, and other pathologies.

Accordingly, in some embodiments, provided herein is a method, comprising: analyzing the presence or amount of CD8+ T Cells in a sample from a subject with a neurodegenerative disorder.

Further embodiments provide a method of characterizing or diagnosing a neurodegenerative disorder, comprising: a) analyzing the presence or amount of CD8+ T Cells in a sample from a subject; and b) identifying the subject as have AD when an increased level of said CD8+ T Cells is present in the sample.

In some embodiments, the method further comprises treating the subject if a neurodegenerative disease is identified. Treatments include, but are not limited to, administration of medication (e.g., cholinesterase inhibitors (e.g., aricept, exalon, razadyne) or memantine (e.g., namenda)); behavior monitoring or modification; treatments for sleep changes; dietary changes; and the like.

In some embodiments, the CD8+ T Cells are CD8+CD45RA+ (TEMRA) cells. In some embodiments, the CD8+ T cell are clonal T cells. In some embodiments, the method further comprises detecting the level of CXCL9 (MIG) in the sample (e.g., blood or blood product sample).

The present disclosure is not limited to particular sample types. In some exemplary embodiments, the sample is blood, plasma or cerebrospinal fluid (CSF). In some embodiments, CD8+ T Cells are detected in CSF and MIG is detected in a blood or plasma sample.

In some embodiments, the subject is a human. In some embodiments, the neurodegenerative disorder is Alzheimer's disease (AD) or Parkinson's disease. In some embodiments, an increased level of CD8+ T Cells in the sample is indicative of the presence of AD in the subject.

The present disclosure in not limited to particular detection methods. In some embodiments, the detecting comprises T cell receptor (TCR) sequencing. In some embodiments, the level of CD8+ T Cells is measured as a percent of all peripheral blood mononuclear cells (PBMCs). In some embodiments, the analyzing comprises one or more of mass cytometry, spanning-tree progression analysis of density-normalized events (SPADE) and/or cluster identification, characterization, and regression (CITRUS) analysis.

Additional embodiments provide a kit, comprising: a) a first reagent for detection of the presence or amount of CD8+ T Cells in a sample from a subject; and/or b) a second reagent for detection of the presence or amount of MIG in a sample from a subject. The kit may include appropriate positive and negative control samples and assay-specific reagents (e.g., buffers).

Certain embodiments provide a kit as described herein for use in characterizing or diagnosing a neurodegenerative disorder.

Yet other embodiments provide the use of a kit as described herein for characterizing or diagnosing a neurodegenerative disorder.

Further embodiments provide a method of treating AD, comprising: a) isolating T Cells from a subject diagnosed with AD; b) engineering the T Cells ex vivo to express a T Cell receptor (TCR) gene from the subject; and c) re-introducing the engineered T Cells into the subject. In some embodiments, the engineered T Cells initiate an immune response against a target (e.g., antigen, protein, or TCR) associated with AD in the brain of said subject. In some embodiments, T Cells (e.g., CD8+ T Cells) are targeted to inhibit their function.

Some embodiments provide an autologous T Cell engineered to express a TCR gene from a subject for use in treating AD in the subject.

Additional embodiments are described herein.

### DESCRIPTION OF THE FIGURES

FIG. 1 shows that mass cytometry of PBMCs reveals an adaptive immune signature of AD.

a) Representative SPADE trees from healthy and MCI/AD patient PBMCs show increased abundance of a CD8⁺ cluster. b) Plotting of clusters by p-value and log 2 fold change reveals cluster 63 as the only significantly increased cluster amongst MCI/AD patients. c) Quantification of individual subjects' cluster 63 show significantly higher percentages of total PBMCs in MCI/AD patients. d) Marker expression analysis of cluster 63 shows this cluster corresponds to a CD3⁺CD8⁺CD45RA⁺CD27⁻ T_{EMRA} population. e) CITRUS clustering (left) showing significant differentiating populations. Cluster 229992 and its significant daughter populations are outlined. g) Marker expression of cluster 229992 shows it to be CD3⁺CD8⁺CD45RA⁺CD27⁻T_{EMRA} population. h) CITRUS' regularized supervised learning algorithm predicts disease group with a 20% error rate (80% positive predictability).

FIG. 2 shows increased stimulatory response in MCI/AD CD8⁺ T cells. a) MCI/AD effector and b) memory CD8⁺ T cells show increased phosphorylation of CREB that is not present in c) naïve CD8⁺ T cells. This same enhancement of MCI/AD CD8+ T cells was observed via increased phosphorylation of ERK in stimulated d) effector and e) memory cells but not f) naïve cells. MFI=mean fluorescence intensity.

FIG. 3 shows association between peripheral CD8⁺ T cells, inflammation and clinical measures in MCI/AD patients. a) Plasma proteomics reveals significantly higher levels of the brain homing chemokine CXCL9 in MCI/AD patient plasma. b) Representative MRI images of an AD brain showing cortical surface rendering, subcortical segmentation and hippocampal segmentation methods used to measure brain volumetry. c) Decreased volumes of hippocampus, subiculum, amygdala and posterior cingulate cortex in MCI/AD patient brains normalized to intracranial volume. d) Hippocampal segmentation shows reduced volumes of CA1 and the molecular layer of the hippocampus. e) CVC plots of healthy and MCI/AD variable sets. f) Plotting cognitive score rₛ values as a normal distribution reveals associations between peripheral CD8⁺ T cells and cognition.

FIG. 4 shows increased TCR clonality in AD CSF CD8⁺ T cells. a) CD3⁺CD8⁺ T cells were analyzed in the cingulate cortex, entorhinal cortex and hippocampus. b) The aged human CSF immune compartment was assessed for prevalence of B cells, innate immune cells and T cells, which showed that the CSF immune compartment is dominated by T cells. c) Among CD3⁺CD8⁺ T cells, T_{EM} and T_{EMRA} are the dominant populations. d) TCR sequencing analysis of CD8⁺ T cells shows increased clonality of AD and PD TCRs compared to age-matched healthy control TCRs. e) Quantification of the clonal proportion of CD8⁺ TCRs reveals greater clonality and f) less diversity in patient TCRs. g) Marker expression analysis of healthy and AD TCRs shows the dominant clone in AD corresponds to a CD3⁺CD8⁺CD45RA⁺CD27⁻T_{EMRA} cell and expresses the CXCL9/brain homing receptor CXCR3. h) Quantification of shared clonotypes within groups shows greater shared clonality amongst diseased patient vs. healthy control TCRs.

FIG. 5 shows study design. a) Patient groups were age-matched and included 57 healthy subjects and 23 MCI/AD patients. b) Quantification of CSF Aβ as ratios to phosphorylated or c) total tau reveals significantly reduced ratios in MCI/AD patients. d) Markers used in this study included 21 cell surface antibodies and DNA interchelators. e) Study design included isolation of PBMCs followed by mass cytomery.

FIG. 6 shows blinded quantification of classical immune variables shows strong correlations with SPADE data. a) Blinded quantification of classical immune variables shows significantly reduced CD4⁺:CD8⁺ T cell ratios, b) increased percentages of CD8⁺ T cells, c) increased prevalence of effector CD8⁺ T cells and d) reduced prevalence of memory CD8⁺ T cells. e) Heatmap showing Spearman correlations between classical immune variables and SPADE clusters reveals strong correlations between CD8⁺ T cell variables and SPADE cluster 63. The table lists the strongest correlates with cluster 63.

FIG. 7 shows reduced senescence of MCI/AD CD8⁺ T_{EMRA} cells. a) SPADE trees were constructed from 10,000 CD8⁺ T cells, which showed a reduced population (cluster 6) in MCI/AD patients versus age-matched (old) controls. b) Marker expression analysis of cluster 6 reveals this cluster to correspond to a senescent CD8⁺ T_{EMRA} cell. c) Significantly lower percentages of cluster 6 in MCI/AD CD8⁺ T cells vs. age-matched, healthy (old) subjects' CD8⁺ T cells (as a percentage of PBMCs).

### DEFINITIONS

To facilitate an understanding of the present disclosure, a number of terms and phrases are defined below:

As used herein, the terms “detect”, “detecting” or “detection” may describe either the general act of discovering or discerning or the specific observation of a composition. Detecting a composition may comprise determining the presence or absence of a composition. Detecting may comprise quantifying a composition. For example, detecting comprises determining the level of a T Cell and/or protein marker. For example, the composition may comprise an antibody that specifically binds to a marker on the T Cell or a protein marker. Alternatively, or additionally, the composition may be a detectably labeled composition (e.g., comprising a label molecule that is distinct from the antibody or other detection composition).

As used herein, the term “subject” refers to any organisms that are screened using the screening and diagnostic methods described herein. Such organisms preferably include, but are not limited to, mammals (e.g., murines, simians, equines, bovines, porcines, canines, felines, and the like), and most preferably includes humans. Alternatively, the organism is an avian, amphibian, reptile or fish.

The term “diagnosed,” as used herein, refers to the recognition of a disease by its signs and symptoms, or genetic analysis, pathological analysis, histological analysis, and the like.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “label” as used herein refers to any atom or molecule that can be used to provide a detectable (preferably quantifiable) effect, and that can be attached to a nucleic acid or protein (e.g., antibody). Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable. In some embodiments, nucleic acids or proteins are detected directly without a label (e.g., directly reading a sequence or based on mass or charge).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present disclosure.

### DETAILED DESCRIPTION OF THE DISCLOSURE

Provided herein are compositions and methods for characterizing neurodegenerative disease and for conducting laboratory tests to analyze samples from subjects (e.g. subjects suspected of having a neurodegenerative disease). In particular, provided herein are compositions and methods for measuring T cell markers associated with Alzheimer's disease. Further embodiments provide compositions and methods for identifying drug targets and drugs for use in treating neurodegenerative disease (e.g., targeted immunotherapy).

### I. Detection of Markers

As described herein, embodiments of the present disclosure provide compositions and methods for detecting marker of neurodegenerative disease (e.g., for research, screening, and diagnostic applications). Exemplary detection methods are described herein.

The present disclosure is not limited to particular sample types. Examples include, but are not limited to, cerebral spinal fluid (CSF), blood, plasma, and the like. In some embodiments, T Cells are detected in CSF and protein markers are detected in blood or plasma.

### A. Detection of T Cells

In some embodiments, provided herein are methods of detecting T Cells (e.g., CD8+ T Cells or CD8+CD45RA+ (TEMRA) cells). In some embodiments, detection comprises cell sorting methods that identify specific T Cells (e.g., CD8+CD45RA+ (TEMRA) cells) in a mixed population of cells. In some embodiments, detection methods determine the percent or ratio of CD8+CD45RA+ (TEMRA) cells or other T Cell of a total population of cells (e.g., peripheral blood mononuclear cells (PBMCs)).

Detection of T Cells is conducted using any one of a number of suitable techniques. For example, cell surface marker expression indicative of a specific population of T Cells (e.g., CD8+CD45RA+ (TEMRA) cells) can be assayed by methods including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, laser capture microdissection, mass cytometry, massively multiparametric mass cytometry, flow cytometry, and FACS analysis.

In some embodiments, T Cells (e.g., CD8+CD45RA+ (TEMRA) cells) are detected by flow cytometry. This method exploits the differential expression of certain surface markers on specific T Cells (e.g., CD8+CD45RA+ (TEMRA) cells) relative to other PBMCs in the sample. Labeled antibodies (e.g., fluorescent antibodies) can be used to react with, or binding agents recognizing other binding agents (e.g., secondary binding agents) that recognize, the markers on cells in the sample for the purpose of enriching or isolating cells by any number of methods including magnetic separation or FACS. In some embodiments, a combination of cell surface markers is utilized in order to further define or quantify the T Cells in the sample in situ (e.g. by immunofluorescence or immunohistochemistry) or using methods analyzing single cells following isolation (e.g. flow cytometry or massively multiparametric mass cytometry). For example, both positive and negative cell sorting may be used to assess various T Cells subpopulations or quantify the amount of T Cells (e.g., CD8+CD45RA+ (TEMRA) cells) in the sample.

In some embodiments, T Cells (e.g., CD8+CD45RA+ (TEMRA) cells) are detected by mass cytometry. Mass cytometry is a newly developed technique for studying biological samples. The technique was originally developed to study cell populations in which samples of interest containing various biological cells were “stained” with affinity probes such as antibodies that are attached to elemental tags. The amount of affinity probes of a given type attached to each cell can be used to characterize each individual cell. The amount of affinity probe of each type is directly related to the amount of the associated elemental tag. The amount of the elemental tagging material can be measured by passing the cell through an inductively coupled plasma (ICP) ion source of a mass cytometer.

In some embodiments, mass cytometry is used in single particle analysis herein where T cells are labeled with metal-conjugated antibodies and metallointercalators and introduced individually into an Inductively Coupled Plasma (ICP) ion source, where the cells are atomized and ionized. The atomic ions are extracted, separated by mass and quantitatively measured in the mass cytometer (MC). The mass cytometer can be, for instance, a mass spectrometer adapted to quantitatively measure the number of each of various different ions per cell. The quantitative measurements for multiple different types of ions can be conducted concurrently, as described in U.S. Pat. No. 7,479,630; herein incorporated by reference in its entirety. The elemental signature of the cell is represented by the element tags associated with the antibodies and metallointercalators. The presence of the metal tag indicates that the antibody conjugated with that tag found and bound the target biomarker, and the intensity of the signal corresponding to that ionized tag is directly proportional to the number of corresponding antibodies bound per cell.

In some embodiments, T Cells are detected using the Luminex technology. In the Luminex technology, following sample preparation aSyn-aggregates recognized by specific aSyn-specific antibodies may be detected by a secondary antibody coupled to fluorescent-dyed microspheres which can be detected in multiplex detecting systems e.g. a Luminex reader (Binder et al., Lupus 15 (2005):412-421).

In some embodiments, immunoassays (e.g., those described below) are utilized to detect T Cells.

In some embodiments, T Cell detection further comprises T Cell sequencing (e.g., as described in the Experimental section). In some embodiments, T Cell sequencing identifies additional biomarkers of neurodegeneration.

### B. Detection of Protein Markers

In some embodiments, protein markers (e.g., MIG) are detected by any suitable method. In some embodiments, proteins are detected by immunohistochemistry. In other embodiments, proteins are detected by their binding to an antibody raised against the protein.

Illustrative non-limiting examples of immunoassays include, but are not limited to: immunoprecipitation; Western blot; ELISA; immunohistochemistry; immunocytochemistry; immunochromatography; flow cytometry; and, immuno-PCR. Polyclonal or monoclonal antibodies detectably labeled using various techniques (e.g., colorimetric, fluorescent, chemiluminescent or radioactive labels) are suitable for use in the immunoassays.

Immunoprecipitation is the technique of precipitating an antigen out of solution using an antibody specific to that antigen. The process can be used to identify proteins or protein complexes present in cell extracts by targeting a specific protein or a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and optionally sorting microscopic particles or cells suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

### C. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present disclosure provides the further benefit that the clinician, who is not likely to be trained in molecular biology, need not understand the raw data. The data is presented directly to the clinician in its most useful form. The clinician is then able to immediately utilize the information in order to optimize the care of the subject.

In some embodiments, data analysis utilizes spanning-tree progression analysis of density-normalized events (SPADE) and/or cluster identification, characterization, and regression (CITRUS) analysis. These data analysis methods are described in more detail in the experimental section below.

The present disclosure contemplates any method capable of receiving, processing, and transmitting the information to and from laboratories conducting the assays, information providers, medical personnel, and subjects. For example, in some embodiments of the present disclosure, a sample (e.g., a CSF or blood or blood product sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., level of one or more markers), specific for the diagnostic or prognostic information desired for the subject.

The profile data is then prepared in a format suitable for interpretation by one or more medical personnel (e.g., a treating clinician, physician assistant, nurse, or pharmacist). For example, rather than providing raw expression data, the prepared format may represent a diagnosis or risk assessment (e.g., presence or absence or level of a marker of neurodegeneration described herein) for the subject, along with recommendations for particular treatment options. The data may be displayed to the medical personnel by any suitable method. For example, in some embodiments, the profiling service generates a report that can be printed for the medical personnel (e.g., at the point of care) or displayed to the medical personnel on a computer monitor.

In some embodiments, the information is first analyzed at the point of care or at a regional facility. The raw data is then sent to a central processing facility for further analysis and/or to convert the raw data to information useful for medical personnel or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the medical personnel, the subject, or researchers.

In some embodiments, the subject is able to directly access the data using the electronic communication system. The subject may choose further intervention or counseling based on the results.

In some embodiments, the data is used for research use. For example, the data may be used to further optimize the inclusion or elimination of markers as useful indicators of a particular condition or stage of disease or as a companion diagnostic to determine a treatment course of action.

### D. Compositions & Kits

Compositions for use in the methods described herein include, but are not limited to, antibodies, reagents, analysis instruments, etc.

The antibody compositions of the present disclosure may also be provided on a solid support. The solid support may comprise one or more beads, plates, solid surfaces, wells, chips, or a combination thereof. The beads may be magnetic, antibody coated, protein A crosslinked, protein G crosslinked, streptavidin coated, oligonucleotide conjugated, silica coated, or a combination thereof. Examples of beads include, but are not limited to, Ampure beads, AMPure XP beads, streptavidin beads, agarose beads, magnetic beads, Dynabeadst, MACS® microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BcMag™ Carboxy-Terminated Magnetic Beads.

The detection reagents can incorporate moieties useful in detection, isolation, purification, or immobilization, if desired. Such moieties are described (see, for example, Ausubel et al., (1997 & updates) Current Protocols in Molecular Biology. Wiley & Sons, New York) and are chosen such that the ability of the probe to hybridize with its target molecule is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

In certain multiplex formats, labels used for detecting different target molecules may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the target molecule. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the disclosure. A variety of different coding schemes are known in the art, including fluorophores, including SCNCs, deposited metals, and RF tags.

Instructions for using the kit to perform one or more methods of the disclosure can be provided, and can be provided in any fixed medium. The instructions may be located inside or outside a container or housing, and/or may be printed on the interior or exterior of any surface thereof. A kit may be in multiplex form for concurrently detecting and/or quantitating one or more different target polynucleotides representing the expressed target molecules.

### II. Screening, Diagnosis, Prognosis, and Monitoring

The methods, compositions, and kits disclosed herein may be used for the characterization, screening, diagnosis, prognosis, and/or monitoring the status or outcome of a neurodegenerative disease (e.g., AD or Parkinson's disease). For example, in some embodiments, individuals identified as having increases level of CD8+ T Cells (e.g., CD8+CD45RA+ (TEMRA) cells) and/or increased levels of protein markers (e.g., MIG) are identified as having AD. In some embodiments, the levels of CD8+ T Cells (e.g., CD8+CD45RA+ (TEMRA) cells) and/or MIG are used to distinguish AD or Parkinson's disease from other neurodegenerative or cognitive disorders. This allows for the correct treatment to be administered to subjects with AD. In addition, subjects not found to have markers indicative of AD can be provided different treatments and/or further diagnostic tests to identify a specific diagnosis.

In some embodiments, diagnosing, predicting, and/or monitoring the status or outcome of a neurodegenerative disease may comprise determining a therapeutic regimen. Determining a therapeutic regimen may comprise administering an anti-neurodegenerative disease therapeutic. Alternatively, determining the treatment for the neurodegenerative disease may comprise modifying a therapeutic regimen. Modifying a therapeutic regimen may comprise increasing, decreasing, or terminating a therapeutic regimen.

In some instances, the methods disclosed herein can diagnose, prognose, and/or monitor the status or outcome of a neurodegenerative disease in a subject with an accuracy of at least about 50%. In other instances, the methods disclosed herein can diagnose, prognose, and/or monitor the status or outcome of a neurodegenerative disease in a subject with an accuracy of at least about 60%. The methods disclosed herein can diagnose, prognose, and/or monitor the status or outcome of a neurodegenerative disease in a subject with an accuracy of at least about 65%. Alternatively, the methods disclosed herein can diagnose, prognose, and/or monitor the status or outcome of a neurodegenerative disease in a subject with an accuracy of at least about 70%. In some instances, the methods disclosed herein can diagnose, prognose, and/or monitor the status or outcome of a neurodegenerative disease in a subject with an accuracy of at least about 75%. In other instances, the methods disclosed herein can diagnose, prognose, and/or monitor the status or outcome of a neurodegenerative disease in a subject with an accuracy of at least about 80%. The methods disclosed herein can diagnose, prognose, and/or monitor the status or outcome of a neurodegenerative disease in a subject with an accuracy of at least about 85%. Alternatively, the methods disclosed herein can diagnose, prognose, and/or monitor the status or outcome of a neurodegenerative disease in a subject with an accuracy of at least about 90%. The methods disclosed herein can diagnose, prognose, and/or monitor the status or outcome of a neurodegenerative disease in a subject with an accuracy of at least about 95%.

The disclosure also encompasses any of the methods disclosed herein where the sensitivity is at least about 45%. In some embodiments, the sensitivity is at least about 50%. In some embodiments, the sensitivity is at least about 55%. In some embodiments, the sensitivity is at least about 60%. In some embodiments, the sensitivity is at least about 65%. In some embodiments, the sensitivity is at least about 70%. In some embodiments, the sensitivity is at least about 75%. In some embodiments, the sensitivity is at least about 80%. In some embodiments, the sensitivity is at least about 85%. In some embodiments, the sensitivity is at least about 90%. In some embodiments, the sensitivity is at least about 95%.

The disclosure also encompasses any of the methods disclosed herein where the level of markers such as CD8+ T Cells (e.g., CD8+CD45RA+ (TEMRA) cells) and/or MIG determines the status or outcome of a neurodegenerative disease in the subject with at least about 45% specificity. In some embodiments, the level of markers determines the status or outcome of a neurodegenerative disease in the subject with at least about 50% specificity. In some embodiments, the level of markers determines the status or outcome of a neurodegenerative disease in the subject with at least about 55%, 60%, 70%, 80%, 85%, 90% or 95% specificity.

In some embodiments, the levels of markers described herein is compared to a reference level in order to determine if the level of a marker is elevated. In some embodiments, the reference level is the level in a subject not diagnosed with or exhibiting symptoms of a neurodegenerative disease. In some embodiments, the level is a population average (e.g., from a group of individuals of similar age or disease status). In some embodiments, the level is the level of a subject of the disclosure prior to symptoms of a neurodegenerative disease.

The compositions and methods described herein further find use in characterizing a sample (e.g., for the presence or level of markers described herein), in research uses (e.g., to understand neurodegenerative diseases), and screening (e.g., drug screening) methods.

Further provided herein are screening methods (e.g., to identify and screen drug targets or candidate drugs). For example, in some embodiments, TCR sequencing identifies candidate drug targets.

### III. Therapeutic Applications

In some embodiments, provided herein are compositions and methods for treating and preventing neurodegenerative disease. In some embodiments, as described above, treatments comprise administering known treatments for AD or Parkinson's disease to individuals identified as having such disorders using the compositions and methods described herein. Treatments for AD include, but are not limited to, administration of medication (e.g., cholinesterase inhibitors (e.g., aricept, exalon, razadyne) or memantine (e.g., namenda)); behavior monitoring or modification; treatments for sleep changes; dietary changes; and the like.

In some embodiments, the present disclosure provides compositions and methods for identifying and providing customized treatment for AD. For example, in some embodiments, TCR from a subject diagnosed with AD are sequenced and used to generate custom T cells that target AD. For example, in some embodiments, T Cells from a subject are isolated and engineered ex vivo to express T Cell receptor genes that target disease-associated antigens. These T Cells are expanded in vitro and then infused into the subject. In some embodiments, such T Cells initiate immune responses (e.g., T Cell mediated immune response) against AD targets in the brain of the subject (e.g., by expressing TCR genes that bind to antigens associated with AD) and initiate an immune response against such antigens.

In some embodiments, T Cells are engineered to target TCRs or antigen targets of such T Cells in the subject (e.g., by engineering T Cells with chimeric antigen receptors). In some embodiments, T cells (e.g. CD8+CD45RA⁺CD27− T cells) from a subject are targeted to be removed or suppressed in their immune function. In some embodiments, these T Cells are targeted via their TCR (e.g. by TCR blockade).

### EXPERIMENTAL

The following examples are provided in order to demonstrate and further illustrate certain preferred embodiments and aspects of the present disclosure and are not to be construed as limiting the scope thereof.

### Example 1

### Methods

### Tissue Collection

Collection of brain tissue, plasma, PBMCs and CSF was approved by the Institutional Review Board of Stanford University, and written consent was obtained from all subjects. Samples were acquired through the NIA funded Stanford Alzheimer's Disease Research Center and the Stanford Brain Rejuvenation Program. Plasma was aliquoted and stored at −80° C. PBMCs were isolated from blood by layering diluted blood (1:1 in PBS) on top of an equal volume of Ficoll, followed by centrifugation and isolation of the buffy coat. CSF was collected by lumbar puncture, then centrifuged at 300G to pellet immune cells. The pellet was then resuspended in Recovery Cell Culture Freezing Medium (MermoFisher). All Samples were frozen overnight at −80° C. and transferred the following day to liquid nitrogen for long-term storage. Brain tissue (Ig per brain region) was acquired under a strict <4 hr post-mortem window, then Dounce homogenized and myelin depleted with anti-myelin beads (Miltenyi) and rinsed with PBS prior to flow cytometry analysis.

### Study Participants

For mass cytometry experiments, PBMCs were analyzed from n=80 study subjects. Of those 80 subjects, 57 were determined to be healthy individuals (average age=73.11 years. ±0.94 SEM) Of the 23 patient samples, 15 were from individuals diagnosed with MCI, and 8 were from individuals diagnosed with AD (average age=71.22 years, +2.04 SEM). Among those who reported ethnicity, the healthy group was comprised of 76.09% White, 13.04% Asian and 10.87% Hispanic subjects. The MCI/AD group was comprised of 63.16% White, 15.79% Asian and 21.05% Hispanic subjects. For cell stimulation, proteomics and MRI brain imaging, a subset of these patients were used. For TCR sequencing, a separate cohort of patients was used. Among these subjects, the average age of healthy controls was 66.4±2.50 SEM; for patients the average age was 66±3.50 SEM.

### Cognitive Testing

Study subjects underwent a battery of neuropsychological assessments to determine group status, including: cognitive examination, evaluation of cerebellar function, deep tendon reflexes, sensory input, and motor function. The Montreal Cognitive Assessment (MoCA)³⁸ examination was used to test study subjects for cognitive impairment. The MoCA assesses several cognitive domains: short-term memory recall (5 points), visuospatial abilities (4), executive functions (4), attention (1), concentration (3), working memory (1), language (6) and orientation to time and space (6). MoCA scores range between 0 and 30. A score of 26 or over is considered to be cognitively typical. For healthy subjects, the average score was 27.55±0.28 SEM. For patients, the average score was 22.55±1.67 SEM.

### Mass Cytometry

Mass cytometry was performed as previously described³⁹. Briefly, cells were thawed in complete medium (RPMI with 10% fetal bovine serum with 1% penicillin-streptomycin) containing 0.1 mg/mL DNase. After washing in Maxpar Cell Staining Buffer (Fluidigm) cells were resuspended in 50 μL filtered antibody cocktail and incubated for 60 min on ice. Cells were again rinsed, then resuspended in 100 μl of 1:3000 diluted In 115-DOTA maleimide in buffer. Following additional rinses, cells were resuspended in 100 μl of 2% paraformaldehyde in buffer and incubated at 4° C. overnight. Cells were then washed twice with permeabilization buffer (eBioscience) and incubated on ice for 45 min. After rinsing, cells were resuspended in Ir-Interchelator in buffer and incubated for 20 min at room temperature. Cells were then washed with buffer, then MilliQ water and finally resuspended in MilliQ water for running on a Helios mass cytometer.

### Cell Stimulation

PBMCs were thawed and plated at a density of 1×10⁶ cells per well in a 24 well plate. After overnight incubation at 37° C., cells were stimulated with a cocktail containing PMA and ionomycin and Brefeldin A (BioLegend) in complete medium. Cells were then incubated an additional 5 hrs before performing intracellular mass cytometry analysis. Mass cytometry was performed as above, only pCREB and pERK antibodies were added in the permeabilization step.

### TCR Amplification by Nested PCR Sequencing

TCR sequencing was conducted according to previously established protocols^{33,40}. Briefly, TCR sequences from live CD3⁺ single cells were obtained by a series of three nested PCR reactions. For all phases of PCR reactions HotStarTaq DNA polymerase (Qiagen) was used. Phase 1 PCR reaction was a multiplexed PCR with multiple Vα and Vβ region primers, Ca and Cβ region primers in a 16 μl reaction. For the Phase 1 PCR reaction, the final concentration of each TCR V-region primer was 0.06 μM and each C-region primer was 0.3 μM. A PCR reaction was done using the following conditions: 95° C. 15 min; 94° C. 30 s, 62° C. 1 min, 72° C. 1 min×16 cycles; 72° C. 10 min; 4° C. Thereafter, a 1 μl aliquot of the Phase 1 product was used as a template for 12-μl Phase 2 PCR reaction. The following cycling conditions were used for Phase 2 PCR: 95° C. 15 min; 94° C. 30 s, 64° C. 1 min, 72° C. 1 min×25 cycles, 72° C. 5 min; 4° C. For the Phase 2 reaction, multiple internally nested TCRVα, TCRVβ, TCRCα and Cβ primers were used (V primers 0.6 M. C primers 0.3 μM). The Phase 2 primers of TCR V-region contained a common 23-base sequence at the 5′ end to enable amplification during the Phase 3 reaction with a common 23-base primer. 1 μl aliquot of the Phase 2 PCR product was used as a template for the 14 μl Phase 3 PCR reaction, which incorporated barcodes and enabled sequencing on the Illumina MiSeq platform. For the Phase 3 PCR reaction, amplification was performed using a 5′ barcoding primer (0.05 IM) containing the common 23-base sequence and a 3′ barcoding primer (0.05 μM) containing sequence of a third internally nested Cα and/or Cβ primer, and Illumina Paired-End primers (0.5 μM each). The following cycling conditions were used for Phase 3 PCR: 95° C. 15 min; 94° C. 30 s, 66° C. 30 s, 72° C. 1 min×25 cycles, 72° C. 5 min; 4° C. The final Phase 3 barcoding PCR reactions for TCRα and TCRβ were done separately. For the Phase 3 reaction, 0.5 μM of the 3′ Cα barcoding primer and the 3′ Cβ barcoding primer were used. In addition to the common 23-base sequence at the 3′ end (that enables amplification of products from the second reaction) and a common 23-base sequence at the 5′ end (that enables amplification with Illumina Paired-End primers), each 5′ barcoding primer contains a unique 5-base barcode that specifies plate and a unique 5-base barcode that specifies row within the plate. These 5′ barcoding primers were added with a multichannel pipette to each of 12 wells within a row within a plate. In addition to the internally nested TCR C-region sequence and a common 23-base sequence at the 3′ end (that enables amplification with Illumina Paired-End primers), each 3′ barcoding primer contained a unique 5-nucleotide barcode that specified column. These 3′ barcoding primers were added with a multichannel pipette to each of eight wells within a column within all plates. After the Phase 3 PCR reaction, each PCR product should had a unique set of barcodes incorporated that specified plate, row and column and had Illumina Paired-End sequences that enabled sequencing on the Illumina MiSeq platform. The PCR products were combined at equal proportion by volume, run on a 1.2% agarose gel, and a band around 350 to 380 bp was excised and gel purified using a Qiaquick gel extraction kit (Qiagen). This purified product was then sequenced.

### TCR Sequencing Analysis

TCR sequencing data was analyzed as previously described^{33,40}. Here, raw sequencing data were processed and demultiplexed using a custom software pipeline to separate reads from every well in every plate as per specified barcodes. All paired ends were assembled by finding a consensus of at least 100 bases in the middle of the read. The resulting paired-end reads were then assigned to wells according to barcode. Primer dimers were filtered out by establishing minimum length of 100 bases for each amplicon. A consensus sequence was obtained for each TCR gene. Because multiple TCR genes might be present in each well, our software establishes a cutoff of >95% sequence identity within a given well. All sequences exceeding 95% sequence identity are assumed to derive from the same TCR gene and a consensus sequence is determined. The 95% cutoff conservatively ensures all sequences derived from the same transcript would be properly assigned.

### TCR Clonality Analysis

Clonality measurements were calculated using the tcR package⁴¹ in R Studio. To get a proportion of clonotypes' sum of reads to the overall number of reads in a repertoire (Σ reads of top clonotypes)/(Σ reads for all clonotypes), the “top.proportion” function was used. To evaluate the diversity of clones, the “repDiversity” function, which measures the ecological diversity index, was used. For determining clonality within groups, amino acid sequences were matched by Hamming distance (two sequences were matched if H≤1) using the “find.clonotypes” function. Here, the logical argument “.norm” was used to perform normalization of the number of shared clonotypes in order to control for variability in cloneset size.

SPADE and CITRUS analyses

SPADE and CITRUS analyses were conducted using Cytobank. For SPADE conducted on mass cytometry data, a target number of nodes of 100 was used for the immunophenotyping assay. This number was based off empirical evaluation of results from multiple runs on the same dataset, which showed comparable results. The SPADE population for immunophenotyping (live CD45⁺ cells) was selected based on the gating strategy shown in FIG. 5. Cells were clustered on all markers except those used to exclude platelets or endothelial cells. For CITRUS, the same population of cells and same clustering channels were used as in SPADE to quantify the abundance of various populations. Results are from 5×10³ events per sample, with a false discovery of 2%, minimum cluster size of 1% and cross validation folds set to 10. The predictive nearest shrunken centroid PAMR association model was used. To avoid spurious results, CITRUS was run with minimum cluster sizes of 1-4%, cross validation folds 5-10 and false discovery rate 1-5% for 1×10⁴, 1.5×10⁴ and 2×10⁵ events, totaling 17 individual runs. For SPADE conducted on flow cytometry data, CD3⁺CD8⁺ cells were gated and clustered with a target number of nodes of 30. Similar predictive ability and statistical significance was observed in several of the CITRUS and SPADE models, which were included in our downstream CVC analysis.

### Brain MRI Imaging

T1 weighted MRI Scans were acquired using Axial 3D fast spoiled gradient sequence (GE Discovery 750). The imaging parameters were optimized for gray/white matter tissue contrast with a repetition time of 5.9 ms, echo time 2 ms, flip angle 15, field-of-view 220 mm, matrix size 256×256, slice thickness 1 mm and 2 NEX. Image analysis was conducted using FreeSurfer 6.0. For subcortical segmentation, the “recon-all” command was used. For hippocampal segmentation, the flag “-hippocampal-subfields-T1” was appended to the “recon-all” command for each patient. To correct for sex differences, all volumetric measurements were normalized to total intracranial volume for each patient.

### CVC Analysis

CVC analysis was conducted using the R package ‘ggraph.’ The analysis is based on calculating pairwise rank correlations between variables. This plot included all 250 variables measured in this study and data from all 80 study subjects, where available. The plot displays a network with nodes representing the variables and lines linking any pairs of variables based on their Spearman rank correlation coefficient with each other. A threshold of |r|>0.7 was used to display only the strongest correlations.

### Flow Cytometry

Flow cytometry was conducted using an LSRFortessa (BD Biosciences). A panel consisting of antibodies conjugated to six different fluorophores was used to classify subsets of memory and senescent T cells. Antibodies used were: CD8α-Pacific blue (BioLegend), CD3-BV650 (BD Biosciences), CD45R0-APC-Cy7 (BioLegend), CCR7-488 (BioLegend), IL-7Rα-PE (BioLegend), CD27-PE-Cy7 (BioLegend). For CSF cell characterization, this same panel was used, but CD19-PE-Cy5 (BioLegend) and CD14-Qdot-705 (ThermoFisher) were included. For sorting CSF T cells for TCR sequencing, the following antibodies were used: CD8α-PE (BioLegend), CD161-PE-Cy7 (BioLegend), CXCR3-APC (BioLegend), CD4-APC-Alexa700 (ThermoFisher), CD39-APC-Cy7 (BioLegend), CD38-FITC (BioLegend), PD-1-BV421 (BD Biosciences), CD45RA-BV605 (BD Biosciences), CD3-BV650 (BD Biosciences), CD27-BV786 (BD Biosciences), CD127-BUV395 (BD Biosciences). For each experiment, a compensation matrix was developed using singly stained and unstained controls or fluorescent beads and all analysis was conducted in Cytobank.

### Proteomics

Inflammatory protein biomarkers were measured using a high-throughput, multiplex “Inflammation” immunoassay (Olink Proteomics) that enabled analysis of 92 inflammation-related proteins across all samples simultaneously. Of the 80 subjects involved in this study, plasma was obtained from 77 of them. Importantly, two internal controls (pooled plasma) were added to monitor the quality of assay performance, as well as the quality of individual samples. The average coefficient of variance among these controls was 3.64%. Data are presented as normalized protein expression values, Olink Proteomics' arbitrary unit on log 2 scale. Quality control was performed in two steps: 1) each sample plate was evaluated on the standard deviation of the internal controls (with a cutoff of <0.2 normalized protein expression) and 2) the quality of each sample was assessed by evaluating the deviation from the median value of the controls for each individual sample. Samples that deviated less than 0.3 normalized protein expression from the median passed quality control. Of the 77 plasma samples measured, 75 passed Olink's quality control, for a success rate of 97%. The average intra-assay coefficient of variance was 8%. Of the 92 analytes measured, 71 were detectable, for a 77% detected protein rate.

### Statistical Methods

All statistical analyses were performed using commercially available software (SPSS or Excel). Comparisons between groups of subjects were performed using multivariate analysis of covariance in cases where there was more than one dependent variable. All experiments were controlled for age and sex as covariates. For cluster analyses, comparisons between groups of subjects were performed using two-way analysis of variance followed by Tukey's test for multiple comparisons. For comparing two groups (in the stimulation assay and TCR clonality/diversity measurements), a two-tailed student's t-test was used. Unless otherwise indicated, a p value of <0.05 was considered statistically significant.

### Results

Neuroinflammation is a pathological hallmark of Alzheimer's disease (AD). Immense effort has been dedicated to understanding innate inflammation in AD, yet little is known about the contribution of the adaptive immune response to the disease. Recent advances in neuroimmunology indicate that soluble circulating factors¹⁻³ and peripheral immunity⁴⁻⁶ play critical roles in brain aging. The brain and spinal cord are surrounded by the meninges, a multipartite membranous covering that contains the cerebrospinal fluid (CSF). The meningeal lymphatic system carries both fluid and immune cells from the CSF, and is connected to the deep cervical lymph nodes, enabling peripheral T cells to respond to brain antigens under certain pathological conditions^{7,8}. Several studies have established that T cells that initially encounter antigen in the periphery can enter the CSF via systemic circulation and patrol the subarachnoid space⁹⁻¹¹. The choroid plexus, which produces the CSF, serves as an interface between the brain and circulation and has been shown to be a site of age-related chronic neuroinflammation in mice^{4,5}. Within the brain itself, a network of perivascular spaces connects with the lymphatic system and provides channels for the efflux of fluid and solutes from the brain interstitial space to the CSF, a system that is impaired in AD¹²⁻¹⁴. Altogether, these advances have challenged basic assumptions in neuroimmunology (e.g. brain tolerance and immune privilege) and the etiology of neurodegenerative diseases.

Aging represents the greatest risk for development of AD¹⁵ and changes in peripheral T cell populations have long been associated with aging and loss of immune competence¹⁶. T cells serve vital functions in conferring immunologic protection by generating effector cells that mediate antigen control and by forming memory cells that provide long-term protective immunity¹⁷. Effector and memory T cells are diversified into distinct subsets with specialized functions, comprising heterogeneous pools of CD4⁺ T helper and CD8⁺ T killer cells. Memory T cells contain populations of central memory (T_{CM}) and effector memory (T_{EM}) cells characterized by distinct homing capacity and effector functionl^{17,18}. While numerous studies have reported distribution¹⁹⁻²¹, function and cytokine secretion²²⁻²⁴ changes in T cells of AD patients, extant studies have yielded conflicting results and generally suffer from the limitations and biases associated with conventional methods or small sample sizes. To circumvent these issues, mass cytometry followed by unbiased discovery and machine learning techniques was used to study the immune repertoires of peripheral blood mononuclear cells (PBMCs) from patients with AD and prodromal mild cognitive impairment (MCI). Importantly, patients were age-matched to cognitively typical, healthy controls (FIG. 5a) and a subset of these patients were verified to have MCI/AD by reduced Aβ:phosphorylated tau (p=9.61×10⁻⁵) and Aβ:total tau (p=0.0002) ratios within the CSF, both indicators of MCI/AD pathology^{25,26} (FIG. 5b-c).

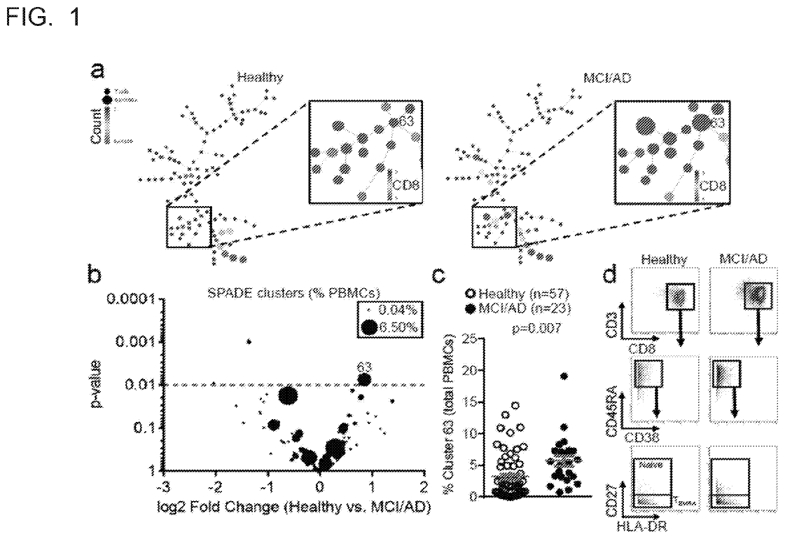

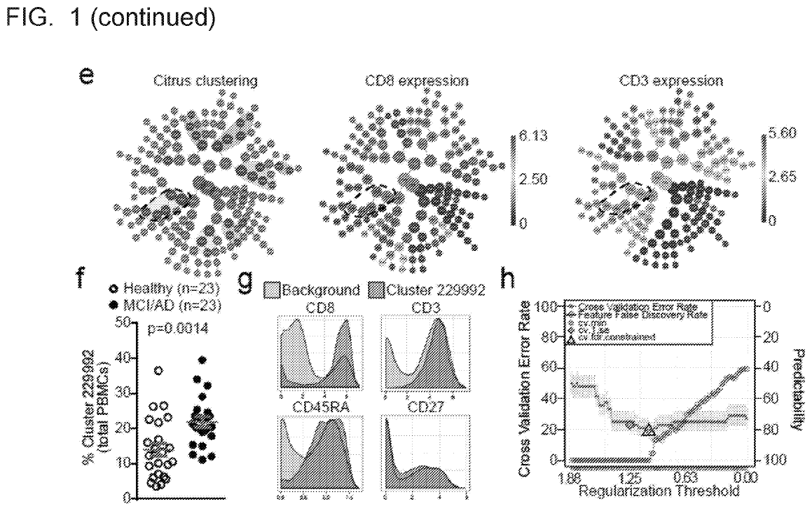

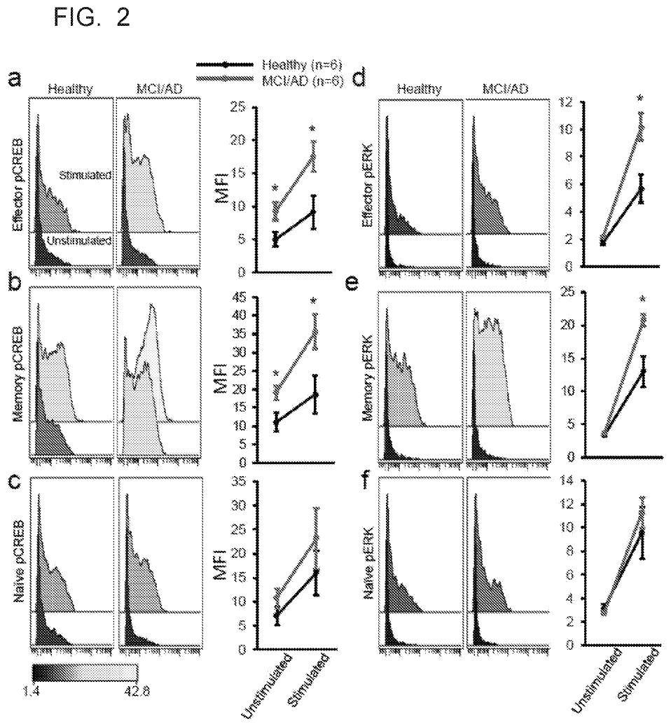

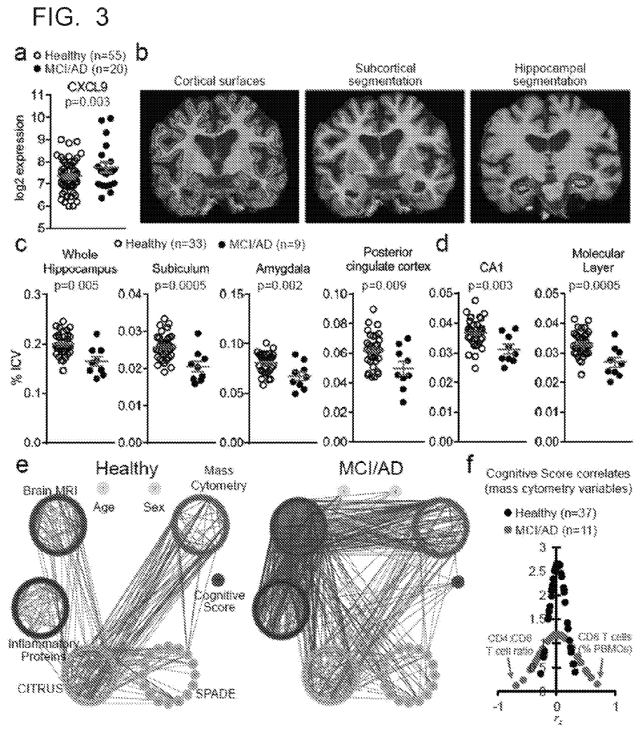

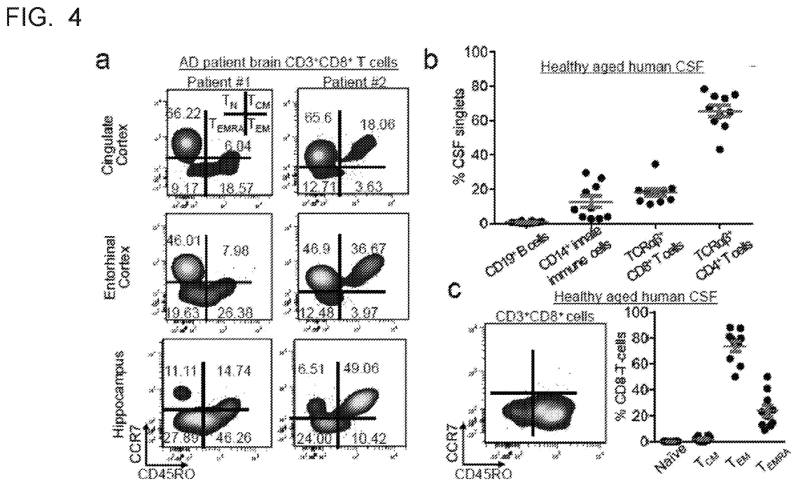

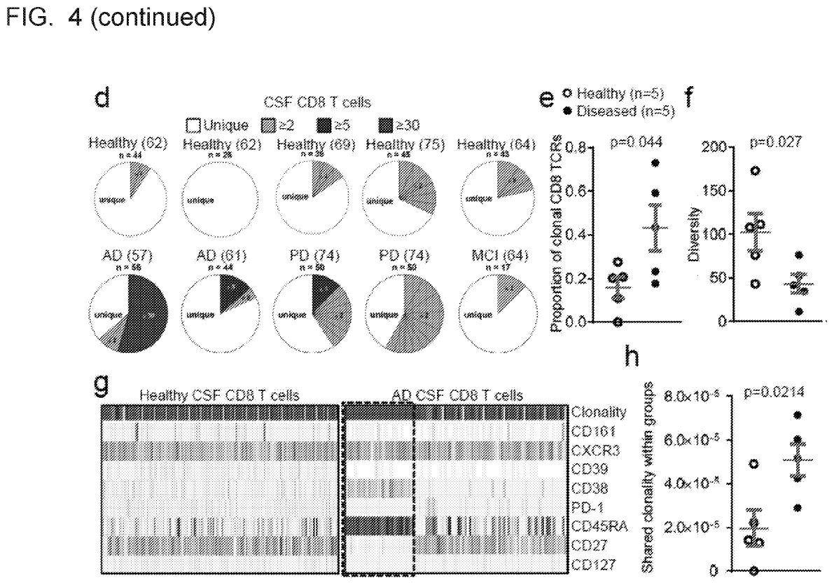

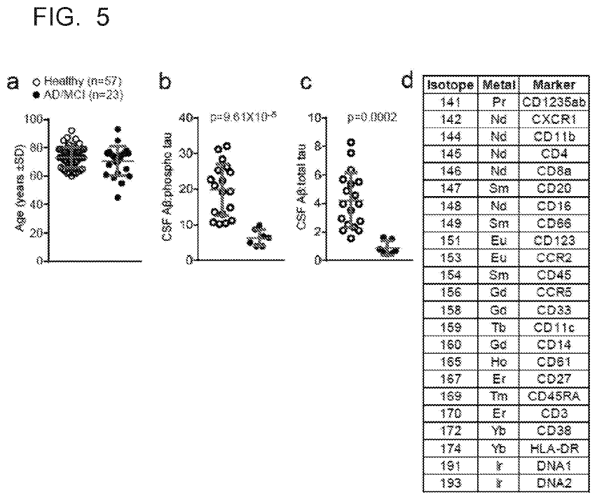

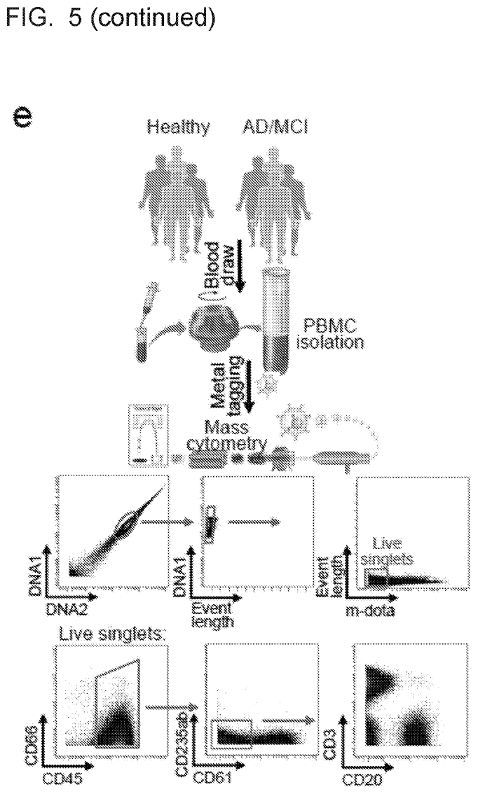

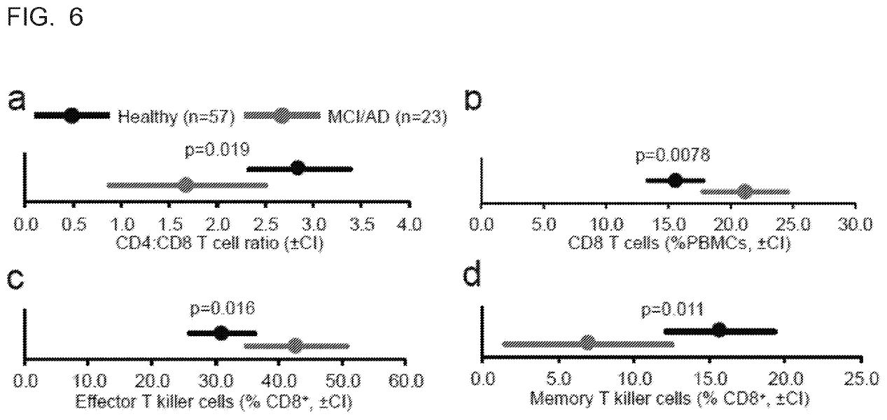

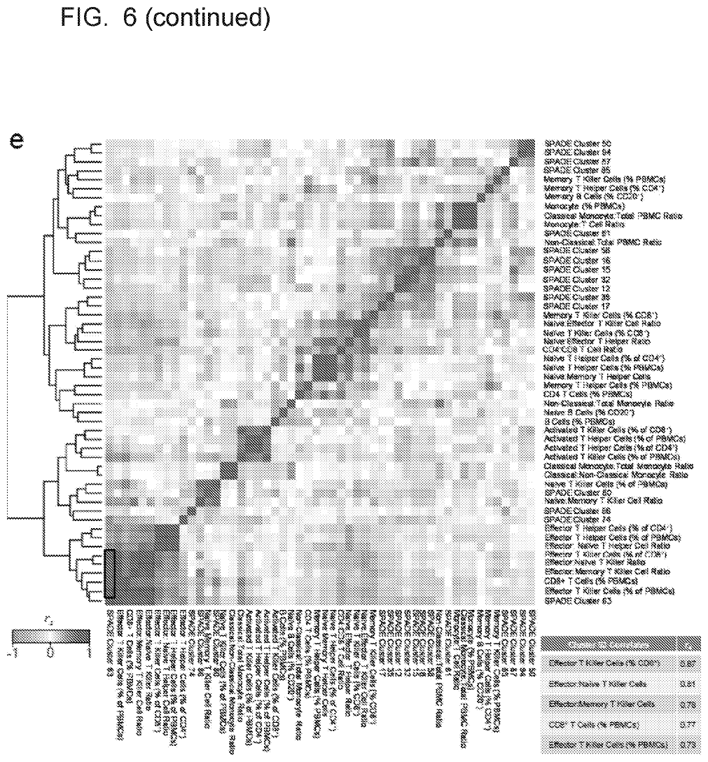

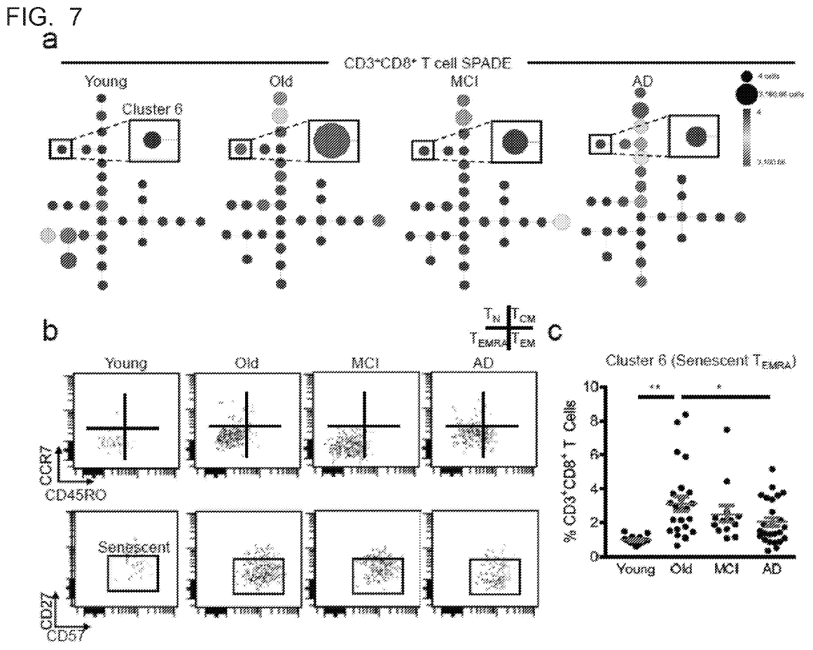

Mass cytometry utilizes heavy metal ion tags to identify antigens²⁷ (as opposed to fluorophores in flow cytometry), allowing for the combination of many more antibody specificities in single samples. Therefore, it was contemplated that this method would be a valuable tool to determine whether MCI/AD patients had an immunologic signature distinct from healthy controls. A panel consisting of 21 immune cell surface markers (FIG. 5d) was used to classify PBMCs from MCI/AD patients and controls (FIG. 5e). This panel allowed for the identification of all major PBMC subsets, including: granulocytes, basophils, plasmacytoid dendritic cells, natural killer cells. T cells, B cells, myeloid dendritic cells, monocytes and platelets. Using Spanning-tree Progression Analysis of Density-normalized Events (SPADE), unsupervised clustering of the multidimensional cytometry data was performed. SPADE trees showed increased representation of a CD8⁺ cluster in patients (cluster 63; FIG. 1a). When plotting all SPADE clusters for p-value vs. fold change, the only significantly increased (p<0.01) cluster among patients was cluster 63 (FIG. 1b). Minute populations that were significantly reduced among patients that corresponded to CD4⁺ T cells were observed (FIG. 1b). Quantification of individual patients' cluster 63 as a percentage of total PBMCs revealed significantly higher values for this cluster in patients (p=0.007; FIG. 1c). Finally, marker expression of cluster 63 demonstrated that this cluster corresponded to CD3⁺CD8⁺CD27⁻ effector memory CD45RA⁺ (T_{EMRA}) cells (FIG. 1d), a highly differentiated T_{EM} population with potent effector functions, including the ability to secrete proinflammatory cytokines and cytotoxic molecules¹⁸.

Cluster identification, characterization, and regression (CITRUS) analysis was used to determine whether clusters could predict disease status. Since machine learning algorithms like CITRUS are most accurate with equal sample sizes, 23 control samples were randomly selected to match patient sample size. Unsupervised hierarchical clustering identified a significantly altered cluster (arbitrarily numbered 229992) corresponding to CD3⁺CD8⁺ T cells (FIG. 1e). Quantification of cluster 229992 (as a percentage of total PBMCs) revealed significantly higher frequency of this population in patient PBMCs (p=0.0036, FIG. 1f). Moreover, marker expression analysis of cluster 229992 again pointed to a population of CD3⁺CD8⁺CD45RA⁺CD27⁻T_{EMRA} cells (FIG. 1g). A regularized supervised learning algorithm was used to determine the populations that best predict whether a given sample belongs to healthy or diseased groups. Cluster 229992 combined with seven additional significantly altered clusters (including CD4⁺ T cells and B cells) was 80% predictive of disease status (FIG. 1h). Taken together, these results show significantly increased numbers of CD8⁺ T_{EMRA} cells in patient blood and indicate alterations in peripheral adaptive immunity in AD.

To validate the unbiased SPADE and CITRUS findings, the mass cytometry dataset was used to blindly quantify 33 immune variables, including ratios of classical/non-classical monocytes, naïve/memory B and T cells and numbers of each cell type as a percentage of total PBMCs (Table 1). Variables that showed significant differences by multivariate analysis of covariance (using age and gender as covariates) between patients and healthy controls were all related to CD8⁺ T cells. Specifically, decreased CD4⁺:CD8⁺ T cell ratios (p=0.019; FIG. 6a) and a concomitant increase in CD8⁺ T cells (as a percentage of PBMCs) in patients vs. controls (p=0.0078; FIG. 6b) were observed. CD8⁺ T cell subsets were also significantly different in patient PBMCs: effector cells were overrepresented (p=0.016; FIG. 6c), while memory cells were underrepresented in patients vs. controls (p=0.011; FIG. 6d). To discover possible interactions between these 33 classical immune variables and the SPADE data, Spearman's rank correlation coefficients were generated and plotted on a heatmap. This analysis revealed strong relationships between SPADE cluster 63 and CD8⁺ T cell variables, including effector T killer cells (as a percentage of CD8⁺ cells; rₛ=0.87), effector:naïve T killer cell ratio (rₛ=0.81), effector:memory T killer cell ratio (rₛ=0.78), CD8⁺ T cells (as a percentage of PBMCs; rₛ=0.77) and effector T killer cells as a percentage of CD8⁺ cells (rₛ=0.73; Extended Data FIG. 2e). To further probe CD8⁺ T cells in MCI/AD, flow cytometry was performed on a separate cohort of patient samples and assessed markers of immunosenescence, an age-associated immune deficiency that leads to reduced proliferative and functional capacity of T cells¹⁶. SPADE diagrams were generated from these data (FIG. 7a), which revealed a significantly reduced cluster of senescent T_{EMRA} cells in AD patients vs. healthy controls (FIG. 7b-c). Cumulatively, these results reveal alterations in peripheral T cell subsets of MCI/AD patients and implicate reduced immunosenescence of the CD8⁺ T_{EMRA} population as cause for increased numbers of these cells in MCI/AD patient blood.

The altered subsets of CD8⁺ T cells in patient PBMCs prompted a test of whether patient CD8⁺ T cells were functionally distinct from controls. To achieve this, a subset of MCI/AD and control PBMCs were stimulated with phorbol 12-myristate 13-acetate (PMA) and ionomycin and used mass cytometry to read out two major downstream signaling pathways: phosphorylation of cAMP response element-binding protein (pCREB) and extracellular signal-regulated kinases (pERK). Activation of these distinct phosphospecies is indicative of signaling events in T cells. Intriguingly, gating of CD8⁺ T cells into naïve, effector and memory subsets revealed significantly higher levels of pCREB in baseline, unstimulated effector and memory cells (FIG. 2a-b), but not naïve cells (FIG. 2c). In PMA stimulated cells, significantly higher levels of pCREB were also detected in MCI/AD vs. control effector and memory cells (FIG. 2a-b), but not naïve cells (FIG. 2c). Phosphorylation of ERK was also significantly increased in stimulated effector and memory cells (FIG. 2d-e), but not naïve cells (FIG. 2f). These results indicate enhanced immune function of patient CD8⁺ T cells that is absent in naïve populations.

The active phenotype of AD CD8⁺ T cells led to an assessment of the immune status of patient blood. Protcomic analysis of immune-related proteins (Table 2) showed significantly increased levels of the T cell trafficking protein C-X-C motif chemokine ligand 9 (CXCL9) in patient plasma (p=0.003; FIG. 3a). Interestingly, CXCL9 is critical for effector T cell homing to the brain of mice through its binding of C-X-C motif chemokine receptor 3 (CXCR3)²⁹⁻³¹. It was next determined whether the peripheral immune changes observed in AD patients were associated with clinical measures. ThreeTesla brain magnetic resonance imaging (MRI) was conducted on a subset of study subjects and these three-dimensional MRI data was analyzed by performing subcortical and hippocampal segmentation followed by volumetric analysis of brain regions (FIG. 3b). The percentage of intracranial volume occupied by numerous brain regions was measured by subcortical segmentation and significant reductions in sizes of patient hippocampus (p=0.005), subiculum (p=0.0005), amygdala (p=0.002) and posterior cingulate cortex were found (p=0.009; FIG. 3c). Hippocampal segmentation also revealed significantly reduced volumes of CA1 and the molecular layer (FIG. 3d), indicative of profound loss of this critical brain region in patients. To depict associations between the clinical data and mass cytometry measurements, a circular visualization of correlation (CVC) plot³ was generated. A CVC plot displays a network with nodes representing groups of variables and lines linking pairs of variables based on their Spearman correlation coefficient. Within this plot, cognitive scores, age, sex, plasma proteomics, brain volumetrics and mass cytometry data, including significant (p<0.05) SPADE and CITRUS clusters was included. CVC analysis revealed a network of associations between variable groups of MCI/AD patients that were absent among healthy participants. In particular, prominent associations between brain volumetric variables and other variable groups—such as plasma proteomics, mass cytometry, and SPADE—were detected among MCI/AD patients (FIG. 3e). Notably, associations between cognitive score and mass cytometry variables were found in MCI/AD patients that did not exist in healthy participants. Plotting of Spearman correlation coefficients between the cognitive score and mass cytometry variables as a normal probability distribution revealed a positive correlation with CD8⁺ T cells as a percentage of total PBMCs (rₛ=0.69) and a negative correlation between the cognitive score and the CD4⁺:CD8⁺ T cell ratio (rₛ=−0.7: FIG. 3f). These data indicate an association between peripheral CD8⁺ T cells, inflammation and disease severity in MCI/AD patients.

The changes observed in peripheral CD8⁺ T cell subsets and their association with clinical measures of disease prompted a study of whether brain regions classically associated with AD contained CD8⁺ memory T cells. The cingulate cortex, entorhinal cortex, and hippocampus of two patient brains were probed by flow cytometry to determine which regions contained CD8⁺ T cells. The predominant CD8⁺ T cell subtype in the cingulate and entorhinal cortex were naïve, while the hippocampus contained almost exclusively memory T cells (FIG. 4a). Since these findings were limited in sample size due to the challenge of obtaining AD brain tissue under a short post-mortem window, central nervous system immunity in the CSF, which can be acquired in living individuals, was measured. The CSF immune compartment is relatively uncharacterized in healthy aged individuals^{11,32}, so percentages of CD19⁺ B cells, CD14⁺ innate immune cells and CD8⁺ and CD4⁺ T cells in ten healthy elderly subjects were measured by flow cytometry. It was found that the healthy aged CSF immune compartment contained a majority of T cells, with a minority of innate immune cells and an undetectable amount of B cells (FIG. 4b). The CD8⁺ T cell repertoire of the aged CSF is comprised exclusively of T_{EM} cells, and T_{EMRA} cells comprise ˜20% of this population (FIG. 4c).

Since T_{EMRA} cells are associated with immunologic memory, it was determined whether AD CSF had clonally expanded T cells, which are characteristic of a specific immune response. TCR sequences are so diverse that they are essentially unique to an individual T cell, so finding two or more T cells with the same TCR sequence is evidence of clonal expansion³³. TCR sequencing was performed on live T cells of healthy controls patients with MCI/AD, as well as patients with Parkinson's disease, a neurodegenerative brain disorder in which antigen-specific T cells were recently discovered in vitro³⁴. Strikingly, AD patient CD8⁺ T cells showed greater TCR clonality than age-matched healthy controls (p=0.044; FIG. 4d-e) and overall TCR populations were less diverse (p=0.027; FIG. 4f). Moreover, marker expression analysis revealed the top AD TCR clone to be expressed by CD8⁺CD45RA⁺CD27⁻ T_{EMRA} cells, which also expressed the CXCL9/brain-homing receptor CXCR3 (FIG. 4f). Finally, within groups, shared clonality was significantly greater amongst neurodegenerative patients than healthy controls (p=0.0214; FIG. 4g). The identification of clonal TCRs in neurodegenerative patients' CSF suggests antigen specificity of these adaptive immune cells. These results are evidence of clonal, antigen-experienced T cells patrolling the intrathecal space in patients with neurodegenerative disease.

Cumulatively, these results shed light on the presence of an adaptive immune response in AD. This is especially pertinent considering that early developmental clinical trials utilizing Aβ vaccination were halted when a portion of phase IIa patients developed aseptic meningoencephalitis driven by brain-infiltrating T cells³⁵⁻³⁷. Furthermore, the existence of clonal CD8⁺ T cells in the CSF of MCI/AD and Parkinson's patients indicates that single cell TCR sequencing may serve as a tool for identifying biomarkers of neurodegeneration.

TABLE 1
Significance of diagnosis (age covariate)

| | | | Marginal | | Interval | |
|---|---|---|---|---|---|---|
| Dependant Variable | value | Diagnosis | Mean | Std. Error | Lower | Upper |
| B cells (% PBMCs) | 0.4574 | Control | 3.366 | .265 | 2.837 | 3.895 |
| | | Diseased | 3.732 | .407 | 2.920 | 4.543 |
| Memory B cells (% CD20⁺) | 0.5245 | Control | 21.305 | 1.820 | 17.679 | 24.930 |
| | | Diseased | 19.161 | 2.792 | 13.599 | 24.724 |
| Naïve B cells (% CD20⁺) | 0.6118 | Control | 65.283 | 2.069 | 61.160 | 69.405 |
| | | Diseased | 67.225 | 3.175 | 60.900 | 73.549 |
| Monocyte (% PBMCs) | 0.6055 | Control | 23.316 | 1.294 | 20.739 | 25.894 |
| | | Diseased | 22.08 | 1.985 | 18.126 | 26.034 |
| Monocyte:T cell ratio | 0.2847 | Control | 0.544 | 0.65 | .435 | .653 |
| | | Diseased | 0.436 | 0.84 | .269 | .602 |
| Classical:Non-classical monocytes ratio | 0.6018 | Control | 7.279 | .534 | 6.216 | 8.343 |
| | | Diseased | 6.764 | .819 | 5.132 | 6.396 |
| Classical monocyte:Total PBMC ratio | 0.5748 | Control | 0.2 | .011 | .178 | .223 |
| | | Diseased | 0.189 | .017 | .154 | .223 |
| Classical:Total monocyte ratio | 0.7189 | Control | 0.847 | .009 | .829 | .865 |
| | | Diseased | 0.853 | .014 | .825 | .881 |
| Non-classical monocytes:total PBMC ratio | 0.7157 | Control | 0.034 | .003 | .029 | .039 |
| | | Diseased | 0.032 | .004 | .024 | .040 |
| Non-classical monocyte:total monocyte ratio | 0.9563 | Control | 0.148 | .009 | .131 | .166 |
| | | Diseased | 0.148 | .013 | .121 | .174 |
| CD4:CD8 T cell ratio | 0.0191* | Control | 2.654 | .265 | 2.326 | 3.382 |
| | | Diseased | 1.685 | .407 | .674 | 2.495 |
| CD4 T cells (% PBMCs) | 0.6010 | Control | 29.718 | 1.198 | 27.332 | 32.104 |
| | | Diseased | 28.56 | 1.838 | 24.899 | 32.221 |
| CD8 T cells (% PBMCs) | 0.0078*** | Control | 15.575 | 1.109 | 13.365 | 17.784 |
| | | Diseased | 21.158 | 1.702 | 17.768 | 24.547 |
| Naïve T helper cells (% CD4⁺) | 0.2163 | Control | 36.869 | 2.382 | 32.124 | 41.613 |
| | | Diseased | 31.4 | 3.654 | 24.121 | 38.679 |
| Memory T helper cells (% CD4⁺) | 0.7531 | Control | 40.782 | 1.748 | 37.299 | 44.264 |
| | | Diseased | 41.796 | 2.682 | 36.455 | 47.141 |
| Effector T helper cells (% of CD4⁺) | 0.1312 | Control | 3.207 | .914 | 1.385 | 5.029 |
| | | Diseased | 5.777 | 1.403 | 2.982 | 8.572 |
| Activated T helper cells (% of CD4⁺) | 0.3818 | Control | 1.036 | .092 | .853 | 1.219 |
| | | Diseased | 1.185 | .141 | .904 | 1.466 |
| Naïve T helper cells (% of PBMCs) | 0.2080 | Control | 12.274 | 1.106 | 10.071 | 14.477 |
| | | Diseased | 9.688 | 1.696 | 6.308 | 13.067 |
| Effector T helper cells (% of PBMCs) | 0.1950 | Control | 0.927 | .316 | .298 | 1.557 |
| | | Diseased | 1.688 | .485 | .722 | 2.654 |
| Activated T helper cells (% of PBMCs) | 0.3015 | Control | 0.278 | .022 | .234 | .322 |
| | | Diseased | 0.321 | .034 | .253 | .389 |
| Memory T helper cells (% of PBMCs) | 0.7581 | Control | 11.58 | .562 | 10.461 | 12.699 |
| | | Diseased | 11.26 | .861 | 9.544 | 12.976 |
| Naïve:Effector T helper cell ratio | 0.0782 | Control | 40.358 | 5.498 | 29.406 | 51.311 |
| | | Diseased | 22.278 | 8.435 | 5.476 | 39.081 |
| Effector:Naïve T helper cell ratio | 0.2797 | Control | 0.171 | .075 | .021 | .321 |
| | | Diseased | 0.322 | .115 | .092 | .552 |
| Naïve:Memory T helper cell ratio | 0.5529 | Control | 1.211 | .200 | .813 | 1.606 |
| | | Diseased | 0.992 | .306 | .382 | 1.601 |
| Naïve T killer cells (% CD8⁺) | 0.8929 | Control | 40.713 | 2.387 | 35.958 | 45.469 |
| | | Diseased | 41.307 | 3.662 | 34.012 | 48.603 |
| Memory T killer cells (% CD8⁺) | 0.0109* | Control | 15.693 | 1.808 | 12.091 | 19.296 |
| | | Diseased | 7 | 2.775 | 1.473 | 12.527 |
| Activated T killer cells (% of CD8⁺) | 0.7099 | Control | 1.775 | .347 | 1.084 | 2.466 |
| | | Diseased | 2.013 | .632 | .954 | 0.075 |
| Effector T killer cells (% of CD8⁺) | 0.0161* | Control | 31.115 | 2.569 | 25.998 | 36.232 |
| | | Diseased | 42.782 | 3.941 | 34.911 | 50.612 |
| Effector T killer cells (% PBMCs) | 0.1385 | Control | 7.626 | 1.143 | 5.348 | 9.904 |
| | | Diseased | 10.779 | 1.754 | 7.285 | 14.274 |
| Activated T killer cells (% PBMCs) | 0.2439 | Control | 0.322 | .065 | .192 | .451 |
| | | Diseased | 0.462 | .099 | .264 | .660 |
| Naïve T killer cells (% PBMCs) | 0.0697 | Control | 6.087 | .611 | 6.069 | 7.105 |
| | | Diseased | 7.619 | .764 | 6.257 | 9.380 |
| Memory T killer cells (% PBMCs) | 0.2449 | Control | 3.576 | .341 | 2.897 | 4.264 |
| | | Diseased | 2.841 | .522 | 1.800 | 3.882 |
| Naïve:effector T killer cell ratio | 0.0296* | Control | 2.356 | .316 | 1.727 | 2.985 |
| | | Diseased | 1.067 | .464 | .102 | 2.032 |
| Naïve:memory T killer cell ratio | 0.0035*** | Control | 2.344 | .327 | 1.692 | 2.995 |
| | | Diseased | 4.164 | .502 | 1.164 | 5.164 |
| Effector:naïve T killer cell ratio | 0.1800 | Control | 1.433 | .278 | .876 | 1.987 |
| | | Diseased | 2.126 | .427 | 1.276 | 2.976 |
| Effector:naïve T killer cell ratio | 0.0007*** | Control | 2.504 | .391 | 1.725 | 3.283 |
| | | Diseased | 5.047 | .600 | 3.852 | 6.243 |
| Granulocytes (% PBMCs) | 0.4180 | Control | 0.154 | .114 | .073 | .380 |
| | | Diseased | 0.017 | .174 | .364 | .331 |
| NK cells (% PBMCs) | 0.9110 | Control | 9.077 | .884 | 7.714 | 10.440 |
| | | Diseased | 8.935 | 1.050 | 6.844 | 11.026 |
| Basophils (% PBMCs) | 0.9160 | Control | 0.148 | .021 | .107 | .190 |
| | | Diseased | 0.144 | .032 | .081 | .207 |
| Plasmacytoid Dendritic cells (% PBMCs) | 0.4430 | Control | 0.635 | .050 | .436 | .634 |
| | | Diseased | 0.464 | .076 | .313 | .616 |
| Myeloid dendritic cells (% PBMCs) | 0.4270 | Control | 0.862 | .052 | .757 | .966 |
| | | Diseased | 0.765 | .080 | .625 | .945 |

TABLE 2
Significance of Diagnosis (age covariate)

| Dependent | | | | Std. | 95% Confidence | |
|---|---|---|---|---|---|---|
| Variable | p-value | Diagnosis | Mean | Error | Lower | Upper |
| ILB | 0.3240 | Healthy | 5.156 | .096 | 4.960 | 5.352 |
| | | MCI/AD | 5.357 | .176 | 5.006 | 5.708 |
| VEGFA | 0.0781 | Healthy | 8.915 | .060 | 8.797 | 9.034 |
| | | MCI/AD | 9.135 | .107 | 8.922 | 9.348 |
| CDCP1 | 0.0053** | Healthy | 2.929 | .067 | 2.796 | 3.063 |
| | | MCI/AD | 3.326 | .119 | 3.088 | 3.564 |
| CD244 | 0.9698 | Healthy | 5.525 | .073 | 5.379 | 5.671 |
| | | MCI/AD | 5.531 | .131 | 5.269 | 5.792 |
| IL7 | 0.3269 | Healthy | 2.869 | .122 | 2.625 | 3.113 |
| | | MCI/AD | 2.62 | .219 | 2.183 | 3.057 |
| OPG | 0.0236 | Healthy | 10.195 | .053 | 10.090 | 10.301 |
| | | MCI/AD | 10.448 | .095 | 10.259 | 10.638 |
| LAP TGF-β1 | 0.6016 | Healthy | 6.682 | .072 | 6.538 | 6.826 |
| | | MCI/AD | 6.72 | .129 | 6.463 | 6.977 |
| uPA | 0.4268 | Healthy | 9.778 | .039 | 9.700 | 9.856 |
| | | MCI/AD | 9.842 | .070 | 9.703 | 9.962 |
| IL-6 | 0.3175 | Healthy | 2.909 | .115 | 2.679 | 3.139 |
| | | MCI/AD | 3.148 | .206 | 2.736 | 3.560 |
| IL-7C | 0.6877 | Healthy | 1.357 | .090 | 1.177 | 1.537 |
| | | MCI/AD | 1.432 | .161 | 1.110 | 1.754 |
| MCP-1 | 0.0521 | Healthy | 9.501 | .057 | 9.386 | 9.614 |
| | | MCI/AD | 9.733 | .102 | 9.530 | 9.935 |
| CXCL11 | 0.1343 | Healthy | 7.253 | .128 | 6.997 | 7.508 |
| | | MCI/AD | 7.653 | .229 | 7.196 | 8.110 |
| AXIN1 | 0.2582 | Healthy | 4.893 | .183 | 4.529 | 5.256 |
| | | MCI/AD | 4.463 | .327 | 3.810 | 5.116 |
| TRAIL | 0.3523 | Healthy | 7.694 | .037 | 7.620 | 7.768 |
| | | MCI/AD | 7.623 | .066 | 7.491 | 7.755 |
| CXCL9 | 0.0025** | Healthy | 7.267 | .104 | 7.060 | 7.475 |
| | | MCI/AD | 7.943 | .186 | 7.571 | 8.315 |
| CST5 | 0.4031 | Healthy | 5.986 | .072 | 5.842 | 6.130 |
| | | MCI/AD | 6.112 | .129 | 5.854 | 6.369 |
| OSM | 0.0289 | Healthy | 2.041 | .095 | 1.851 | 2.230 |
| | | MCI/AD | 2.478 | .170 | 2.139 | 2.817 |
| CXCL1 | 0.4701 | Healthy | 8.005 | .138 | 7.730 | 8.260 |
| | | MCI/AD | 7.798 | .247 | 7.305 | 8.290 |
| CCL4 | 0.7935 | Healthy | 5.896 | .089 | 5.718 | 6.073 |
| | | MCI/AD | 5.944 | .159 | 5.626 | 6.261 |
| CD6 | 0.4486 | Healthy | 3.955 | .099 | 3.758 | 4.153 |
| | | MCI/AD | 4.111 | .177 | 3.757 | 4.465 |
| SCF | 0.1807 | Healthy | 9.491 | .051 | 9.389 | 9.594 |
| | | MCI/AD | 9.635 | .092 | 9.451 | 9.819 |
| IL-18 | 0.5324 | Healthy | 7.175 | .071 | 7.034 | 7.315 |
| | | MCI/AD | 7.266 | .126 | 7.014 | 7.518 |
| SLAMF1 | 0.0208 | Healthy | 1.289 | .050 | 1.190 | 1.388 |
| | | MCI/AD | 1.532 | .069 | 1.354 | 1.709 |
| TGF-α | 0.0044** | Healthy | 2.222 | .033 | 2.156 | 2.288 |
| | | MCI/AD | 2.423 | .059 | 2.305 | 2.541 |
| MCP-4 | 0.6137 | Healthy | 3.29 | .104 | 3.062 | 3.498 |
| | | MCI/AD | 3.593 | .186 | 3.221 | 3.965 |
| CCL11 | 0.0082** | Healthy | 7.194 | .051 | 7.093 | 7.295 |
| | | MCI/AD | 7.479 | .091 | 7.298 | 7.660 |
| TNFSF14 | 0.7493 | Healthy | 3.885 | .115 | 3.655 | 4.115 |
| | | MCI/AD | 3.961 | .207 | 6.549 | 4.374 |
| FGF23 | 0.8723 | Healthy | 2.386 | .055 | 2.276 | 2.497 |
| | | MCI/AD | 2.405 | .099 | 2.207 | 2.602 |
| IL-10RA | 0.9911 | Healthy | 0.987 | .138 | .711 | 1.262 |
| | | MCI/AD | 0.983 | .247 | .490 | 1.477 |
| FGF-5 | 0.0415 | Healthy | 0.859 | .041 | .776 | .941 |
| | | MCI/AD | 1.036 | .074 | .889 | 1.184 |
| MMP1 | 0.5872 | Healthy | 12.155 | .157 | 11.842 | 12.468 |
| | | MCI/AD | 12.331 | .281 | 11.771 | 12.892 |
| LIF-R | 0.0331 | Healthy | 2.447 | .038 | 2.370 | 2.523 |
| | | MCI/AD | 2.619 | .089 | 2.482 | 2.756 |
| FGF-21 | 0.4500 | Healthy | 5.167 | .160 | 4.848 | 5.485 |
| | | MCI/AD | 4.916 | .286 | 4.346 | 5.487 |
| CCL19 | 0.7630 | Healthy | 8.061 | .114 | 7.833 | 8.289 |
| | | MCI/AD | 7.99 | .205 | 7.561 | 8.398 |
| IL-15RA | 0.0516 | Healthy | 0.619 | .026 | .567 | .670 |
| | | MCI/AD | 0.725 | .046 | .632 | .817 |
| IL-10RB | 0.224 | Healthy | 6.315 | .037 | 6.242 | 6.388 |
| | | MCI/AD | 6.492 | .066 | 6.361 | 6.622 |
| IL-18R1 | 0.7367 | Healthy | 6.404 | .065 | 6.274 | 6.534 |
| | | MCI/AD | 6.359 | .117 | 6.126 | 6.592 |
| IL-15RA | 0.0516 | Healthy | 0.619 | .026 | .567 | .670 |
| | | MCI/AD | 0.725 | .046 | .632 | .817 |
| IL-10RB | 0.0224 | Healthy | 6.315 | .037 | 6.242 | 6.388 |
| | | MCI/AD | 6.492 | .066 | 6.361 | 6.622 |
| IL-18R1 | 0.7367 | Healthy | 6.404 | .065 | 6.274 | 6.534 |
| | | MCI/AD | 6.359 | .117 | 6.126 | 6.592 |
| PD-L1 | 0.1397 | Healthy | 3.484 | .053 | 3.378 | 3.591 |
| | | MCI/AD | 3.649 | .096 | 3.458 | 3.640 |
| NGFβ | 0.3444 | Healthy | 1.296 | .036 | 1.226 | 1.369 |
| | | MCI/AD | 1.366 | .064 | 1.240 | 1.496 |
| CXCL5 | 0.6091 | Healthy | 10.054 | .205 | 9.644 | 10.464 |
| | | MCI/AD | 9.836 | .368 | 9.102 | 10.570 |
| TRANCE | 0.5458 | Healthy | 3.496 | .076 | 3.347 | 3.650 |
| | | MCI/AD | 3.403 | .136 | 3.132 | 3.674 |
| HGF | 0.0690 | Healthy | 7.668 | .052 | 7.464 | 7.672 |
| | | MCI/AD | 7.767 | .093 | 7.580 | 7.953 |
| IL-12B | 0.0179 | Healthy | 3.572 | .077 | 3.418 | 3.725 |
| | | MCI/AD | 3.957 | .138 | 3.682 | 4.231 |
| MMP-10 | 0.1931 | Healthy | 5.634 | .083 | 5.468 | 5.800 |
| | | MCI/AD | 5.86 | .149 | 5.563 | 6.158 |
| IL-10 | 0.8166 | Healthy | 2.261 | .128 | 2.006 | 2.517 |
| | | MCI/AD | 2.2 | .229 | 1.743 | 2.657 |
| CCL23 | 0.0463 | Healthy | 9.103 | .070 | 8.963 | 9.242 |
| | | MCI/AD | 9.393 | .125 | 9.143 | 9.642 |
| CD5 | 0.0579 | Healthy | 4.269 | .051 | 4.186 | 4.391 |
| | | MCI/AD | 4.493 | .092 | 4.310 | 4.676 |
| CCL3 | 0.3116 | Healthy | 4.463 | .081 | 4.292 | 4.614 |
| | | MCI/AD | 4.623 | .145 | 4.335 | 4.912 |
| Fit3L | 0.4050 | Healthy | 8.332 | .051 | 8.230 | 8.435 |
| | | MCI/AD | 8.421 | .092 | 8.238 | 8.605 |
| CXCL6 | 0.6277 | Healthy | 8.79 | .131 | 6.529 | 7.051 |
| | | MCI/AD | 6.731 | .234 | 6.263 | 7.198 |
| CXCL10 | 0.0740 | Healthy | 8.395 | .102 | 8.192 | 8.598 |
| | | MCI/AD | 8.776 | .182 | 8.413 | 9.139 |
| EBP1 | 0.8175 | Healthy | 8.061 | .133 | 7.796 | 8.326 |
| | | MCI/AD | 8.125 | .238 | 7.650 | 8.599 |
| SIRT2 | 0.6154 | Healthy | 3.873 | .179 | 3.517 | 4.230 |
| | | MCI/AD | 3.787 | .320 | 3.148 | 4.426 |
| CCL28 | 0.001** | Healthy | 1.333 | .040 | 1.254 | 1.413 |
| | | MCI/AD | 1.609 | .071 | 1.466 | 1.752 |
| DNER | 0.2588 | Healthy | 7.51 | .038 | 7.434 | 7.566 |
| | | MCI/AD | 7.599 | .068 | 7.464 | 7.735 |
| EN-RAGE | 0.0858 | Healthy | 1.179 | .075 | 1.029 | 1.330 |
| | | MCI/AD | 1.451 | .135 | 1.181 | 1.720 |
| CD40 | 0.6632 | Healthy | 9.848 | .090 | 9.668 | 10.027 |
| | | MCI/AD | 9.929 | .161 | 9.607 | 10.251 |
| FGF19 | 0.4545 | Healthy | 7.291 | .116 | 7.060 | 7.522 |
| | | MCI/AD | 7.471 | .208 | 7.057 | 7.685 |
| MCP2 | 0.1840 | Healthy | 7.439 | .069 | 7.261 | 7.618 |
| | | MCI/AD | 7.687 | .160 | 7.368 | 8.006 |
| CASP8 | 0.3459 | Healthy | 1.545 | .091 | 1.363 | 1.727 |
| | | MCI/AD | 1.724 | .164 | 1.398 | 2.050 |
| CCL25 | 0.008** | Healthy | 5.546 | .074 | 5.397 | 5.694 |
| | | MCI/AD | 5.965 | .133 | 5.700 | 6.231 |
| CX3CL1 | 0.2361 | Healthy | 5.1 | .050 | 5.000 | 5.199 |
| | | MCI/AD | 5.223 | .089 | 5.045 | 5.402 |
| TNFRSF9 | 0.0718 | Healthy | 5.543 | .057 | 5.430 | 5.656 |
| | | MCI/AD | 5.756 | .101 | 5.554 | 5.959 |
| NT-3 | 0.5804 | Healthy | 1.63 | .046 | 1.538 | 1.722 |
| | | MCI/AD | 1.683 | .083 | 1.518 | 1.849 |
| TWEAK | 0.2637 | Healthy | 8.664 | .044 | 8.575 | 6.752 |
| | | MCI/AD | 8.767 | .079 | 6.609 | 8.925 |
| CCL20 | 0.3492 | Healthy | 4.682 | .160 | 4.364 | 5.000 |
| | | MCI/AD | 4.992 | .266 | 4.422 | 5.562 |
| ST1A1 | 0.4643 | Healthy | 2.756 | .126 | 2.504 | 3.007 |
| | | MCI/AD | 2.564 | .226 | 2.114 | 3.014 |
| STAMPB | 0.6169 | Healthy | 5.515 | .166 | 5.183 | 5.846 |
| | | MCI/AD | 5.435 | .298 | 4.841 | 6.029 |
| ADA | 0.3960 | Healthy | 3.448 | .067 | 3.315 | 3.561 |
| | | MCI/AD | 3.566 | .120 | 3.327 | 3.804 |
| TNFB | 0.4084 | Healthy | 3.138 | .048 | 3.042 | 3.234 |
| | | MCI/AD | 3.221 | .086 | 3.049 | 3.393 |
| CSF-1 | 0.0361 | Healthy | 7.292 | .034 | 7.223 | 7.360 |
| | | MCI/AD | 7.442 | .062 | 7.319 | 7.565 |

### REFERENCES

1 Villeda, S. A. et al. The ageing systemic milieu negatively regulates neurogenesis and cognitive function. Nature 477, 90-94, doi:10.1038/nature10357 (2011).
2 Villeda, S. A. & Wyss-Coray, T. The circulatory systemic environment as a modulator of neurogenesis and brain aging. Autoimmun Rev 12, 674-677, doi:10.1016/j.autrev.2012.10.014 (2013).
3 Lehallier, B. et al. Combined Plasma and Cerebrospinal Fluid Signature for the Prediction of Midterm Progression From Mild Cognitive Impairment to Alzheimer Disease. JAMA Neurol 73, 203-212, doi:10.1001/jamaneurol.2015.3135 (2016).
4 Schwartz, M. Can immunotherapy treat neurodegeneration? Science 357, 254-255, doi:10.1126/science.aai8231 (2017).
5 Baruch, K. et al. Aging. Aging-induced type I interferon response at the choroid plexus negatively affects brain function. Science 346, 89-93, doi:10.1126/science.1252945 (2014).
6 Baruch, K. et al. PD-1 immune checkpoint blockade reduces pathology and improves memory in mouse models of Alzheimer's disease. Nat Med 22, 135-137, doi:10.1038/nm.4022 (2016).
7 Louveau, A. et al. Structural and functional features of central nervous system lymphatic vessels. Nature 523, 337-341, doi:10.1038/nature14432 (2015).
8 Aspelund, A. et al. A dural lymphatic vascular system that drains brain interstitial fluid and macromolecules. J Exp Med 212, 991-999, doi:10.1084/jem.20142290 (2015).
9 Bartholomaus, I. et al. Effector T cell interactions with meningeal vascular structures in nascent autoimmune CNS lesions. Nature 462, 94-98, doi:10.1038/nature08478 (2009).
10 Reboldi, A. et al. C—C chemokine receptor 6-regulated entry of TH-17 cells into the CNS through the choroid plexus is required for the initiation of EAE. Nat Immunol 10, 514-523, doi:10.1038/ni.1716 (2009).
11 Kivisakk, P. et al. Human cerebrospinal fluid central memory CD4+ T cells: evidence for trafficking through choroid plexus and meninges via P-selectin. Proc Nad Acad Sci USA 100, 8389-8394, doi:10.1073/pnas.1433000100 (2003).
12 Xie, L. et al. Sleep drives metabolite clearance from the adult brain. Science 342, 373-377, doi:10.1126/science.1241224 (2013).
13 Yang, L. et al. Evaluating glymphatic pathway function utilizing clinically relevant intrathecal infusion of CSF tracer. J Transl Med 11, 107, doi:10.1186/1479-5876-11-107 (2013).
14 Iliff, J. J. et al. A paravascular pathway facilitates CSF flow through the brain parenchyma and the clearance of interstitial solutes, including amyloid beta. Sci Transl Med 4, 147ra111, doi:10.1126/scitranslmed.3003748 (2012).
15 Wyss-Coray, T. Ageing, neurodegeneration and brain rejuvenation. Nature 539, 180-186, doi:10.1038/nature20411 (2016).
16 Nikolich-Zugich, J. The twilight of immunity: emerging concepts in aging of the immune system. Nat Immunol 19, 10-19, doi:10.1038/s41590-017-0006-x (2018).
17 Sallusto, F., Lenig, D., Forster. R., Lipp, M. & Lanzavecchia, A. Two subsets of memory T lymphocytes with distinct homing potentials and effector functions. Nature 401, 708-712, doi:10.1038/44385 (1999).
18 Sallusto, F., Geginat, J. & Lanzavecchia, A. Central memory and effector memory T cell subsets: function, generation, and maintenance. Annu Rev Immunol 22, 745-763, doi:10.1146/annurev.immunol.22.012703.104702 (2004).
19 Tan, J. et al. CD45 isoform alteration in CD4+ T cells as a potential diagnostic marker of Alzheimer's disease. J Neuroimmunol 132, 164-172 (2002).
20 Togo, T. et al. Occurrence of T cells in the brain of Alzheimer's disease and other neurological diseases. J Neuroimmunol 124, 83-92 (2002).
21 Lombardi, V. R., Garcia, M., Rey, L. & Cacabelos, R. Characterization of cytokine production, screening of lymphocyte subset patterns and in vitro apoptosis in healthy and Alzheimer's Disease (AD) individuals. J Neuroimmunol 97, 163-171 (1999).
22 Bongioanni, P., Boccardi, B., Borgna, M., Castagna, M. & Mondino, C. T-cell interferon gamma binding in patients with dementia of the Alzheimer type. Arch Neurol 54, 457-462 (1997).
23 Monsonego, A. et al. Increased T cell reactivity to amyloid beta protein in older humans and patients with Alzheimer disease. J Clin Invest 112, 415-422, doi:10.1172/JCI18104 (2003).
24 Monsonego, A., Imitola, J., Zota, V., Oida, T. & Weiner, H. L. Microglia-mediated nitric oxide cytotoxicity of T cells following amyloid beta-peptide presentation to Th1 cells. J Immunol 171, 2216-2224 (2003).
25 Herukka, S. K., Hallikainen, M., Soininen, H. & Pirttila, T. CSF Abeta42 and tau or phosphorylated tau and prediction of progressive mild cognitive impairment. Neurology 64, 1294-1297, doi:10.1212/01.WNL.0000156914.16988.56 (2005).
26 Blennow, K. & Hampel, H. CSF markers for incipient Alzheimers disease. Lancer Neurol 2, 605-613 (2003).
27 Bandura, D. R. et al. Mass cytometry: technique for real time single cell multitarget immunoassay based on inductively coupled plasma time-of-flight mass spectrometry. Anal Chem 81, 6813-6822, doi:10.1021/ac901049w (2009).
28 Bruggner, R. V., Bodenmiller, B., Dill, D. L., Tibshirani, R. J. & Nolan, G. P. Automated identification of stratifying signatures in cellular subpopulations. Proc Natl Acad Sci USA 111, E2770-2777, doi:10.1073/pnas.1408792111 (2014).
29 Miu, J. et al. Chemokine gene expression during fatal murine cerebral malaria and protection due to CXCR3 deficiency. J Immunol 180, 1217-1230 (2008).
30 Campanella, G. S. et al. Chemokine receptor CXCR3 and its ligands CXCL9 and CXCL10 are required for the development of murine cerebral malaria. Proc Natl Acad Sci USA 105, 4814-4819, doi:10.1073/pnas.0801544105 (2008).
31 Ochiai, E. et al. CXCL9 is important for recruiting immune T cells into the brain and inducing an accumulation of the T cells to the areas of tachyzoite proliferation to prevent reactivation of chronic cerebral infection with Toxoplasma gondii. Am J Pathol 185, 314-324, doi:10.1016/j.ajpath.2014.10.003 (2015).
32 Giunti, D. et al. Phenotypic and functional analysis of T cells homing into the CSF of subjects with inflammatory diseases of the CNS. J Leukoc Biol 73, 584-590 (2003).
33 Han, A., Glanville, J., Hansmann, L. & Davis, M. M. Linking T-cell receptor sequence to functional phenotype at the single-cell level. Nat Biotechnol 32, 684-692, doi:10.1038/nbt.2938 (2014).
34 Sulzer, D. et al. T cells from patients with Parkinson's disease recognize alpha-synuclein peptides. Nature 546, 656-661, doi:10.1038/nature22815 (2017).
35 Nicoll, J. A. et al. Neuropathology of human Alzheimer disease after immunization with amyloid-beta peptide: a case report. Nat Med 9, 448452, doi:10.1038/nm840 (2003).
36 Check, E. Nerve inflammation halts trial for Alzheimers drug. Nature 415, 462, doi:10.1038/415462a (2002).
37 Pfeifer, M. et al. Cerebral hemorrhage after passive anti-Abeta immunotherapy. Science 298, 1379, doi:10.1126/science.1078259 (2002).
38 Nasreddine, Z. S. et al. The Montreal Cognitive Assessment, MoCA: a brief screening tool for mild cognitive impairment. J Am Geriatr Soc 53, 695-699, doi:10.1111/j.1532-5415.2005.53221.x (2005).
39 Leipold, M. D., Newell, E. W. & Maecker, H. T. Multiparameter Phenotyping of Human PBMCs Using Mass Cytometry. Methods Mol Biol 1343, 81-95, doi:10.1007/978-1-4939-2963-4_7 (2015).
40 Han, A. et al. Dietary gluten triggers concomitant activation of CD4+ and CD8+ alphabeta T cells and gammadelta T cells in celiac disease. Proc Natl Acad Sci USA 110, 13073-13078. doi:10.1073/pnas.1311861110 (2013).
41 Nazarov, V. I. et al. tcR: an R package for T cell receptor repertoire advanced data analysis. BMC Bioinformatics 16, 175, doi:10.1186/s12859-015-0613-1 (2015).

All publications, patents, patent applications and accession numbers mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

We claim:

1. A method comprising:
analyzing the presence or amount of CD8+ T Cells in a sample from a subject with a neurodegenerative disorder.

2. The method of claim 1, wherein said CD8+ T Cells are CD8+CD45RA+ (TEMRA) cells.

3. The method of claim 1 or 2, wherein said CD8+ T cell are clonal T cells.

4. The method of any one of the preceding claims, wherein said sample is selected from the group consisting of blood, plasma and cerebrospinal fluid (CSF).

5. The method of any one of the preceding claims, wherein said subject is a human.

6. The method of any one of the preceding claims, wherein said neurodegenerative disorder is Alzheimer's disease (AD) or Parkinson's disease.

7. The method of any one of the preceding claims, wherein an increased level of said CD8+ T Cells in said sample is indicative of the presence of AD in said subject.

8. The method of any one of the preceding claims, wherein said detecting comprises T cell receptor (TCR) sequencing.

9. The method of any one of the preceding claims, wherein the level of said CD8+ T Cells is measured as a percent of all peripheral blood mononuclear cells (PBMCs).

10. The method of any one of the preceding claims, wherein said method further comprises detecting the level of CXCL9 (MIG) in said sample.

11. The method of any one of the preceding claims, wherein said CD8+ T Cells are detected in a CSF sample and said MIG is detected in a plasma sample.

12. The method of any one of the preceding claims, wherein said analyzing comprises mass cytometry.

13. The method of any one of the preceding claims, wherein said analyzing comprises spanning-tree progression analysis of density-normalized events (SPADE) and/or cluster identification, characterization, and regression (CITRUS) analysis.

14. A method of characterizing or diagnosing a neurodegenerative disorder, comprising:
a) analyzing the presence or amount of CD8+ T Cells in a sample from a subject; and
b) identifying said subject as have AD when an increased level of said CD8+ T Cells is present in said sample.

15. The method of claim 14, wherein said CD8+ T Cells are CD8+CD45RA+ (TEMRA) cells.

16. The method of claim 14 or 15, wherein said CD8+ T cell are clonal T cells.

17. The method of any one of the preceding claims, wherein said sample is selected from the group consisting of blood, plasma and cerebrospinal fluid (CSF).

18. The method of any one of the preceding claims, wherein said subject is a human.

19. The method of any one of the preceding claims, wherein said neurodegenerative disorder is Alzheimer's disease (AD) or Parkinson's disease.

20. The method of any one of the preceding claims, wherein an increased level of said CD8+ T Cells in said sample is indicative of the presence of AD in said subject.

21. The method of any one of the preceding claims, wherein said detecting comprises T cell receptor (TCR) sequencing.

22. The method of any one of the preceding claims, wherein the level of said CD8+ T Cells is measured as a percent of all peripheral blood mononuclear cells (PBMCs).

23. The method of any one of the preceding claims, wherein said method further comprises detecting the level of CXCL9 (MIG) in said sample.

24. The method of any one of the preceding claims, wherein said CD8+ T Cells are detected in a CSF sample and said MIG is detected in a plasma sample.

25. The method of any one of the preceding claims, wherein said analyzing comprises mass cytometry.

26. The method of any one of the preceding claims, wherein said analyzing comprises spanning-tree progression analysis of density-normalized events (SPADE) and/or cluster identification, characterization, and regression (CITRUS) analysis.

27. A method of characterizing or diagnosing a neurodegenerative disorder, comprising:
a) having a sample from a subject tested for the presence or amount of CD8+ T Cells; and
b) treating said subject for AD when an increased level of said CD8+ T Cells is present in said sample and not treating said subject for AD when an increased level of said CD8+ T Cells is not present in said sample.

28. The method of claim 27 wherein said treatment for AD disease is selected from the group consisting of medication, dietary changes, and behavior modification.

29. The method of claim 28, wherein said medication is selected from the group consisting of cholinesterase inhibitors and memantine.

30. The method of claim 29, wherein said cholinesterase inhibitor is selected from the group consisting of aricept, exalon, and razadyne and said memantine is Namenda.

31. The method of any one of claims 27 to 30, further comprising repeating said having a sample tested.

32. A kit, comprising:
a) a first reagent for detection of the presence or amount of CD8+ T Cells in a sample from a subject; and
b) a second reagent for detection of the presence or amount of MIG in a sample from a subject.

33. The kit of claim 32 for use in characterizing or diagnosing a neurodegenerative disorder.

34. The use of the kit of claim 32 for characterizing or diagnosing a neurodegenerative disorder.

35. A method of treating AD, comprising:
a) isolating T Cells from a subject diagnosed with AD;
b) engineering said T Cells ex vivo to express a T Cell receptor (TCR) gene from said subject; and
c) re-introducing said engineered T Cells into said subject.

36. The method of claim 35, wherein said TCR gene is from a TCR that binds to CD8+ T Cells.

37. The method of claim 36, wherein said CD8+ T Cells are CD8+CD45RA+ (TEMRA) cells.

38. The method of claim 35, wherein said engineered T Cells initiate an immune response against a target associated with AD in the brain of said subject.

39. The method of claim 35, wherein said engineered T Cells initiate an immune response against a TCR associated with AD in the brain of said subject.

40. An autologous T Cell engineered to express a TCR gene from a subject for use in treating AD in said subject.
